(19) United States
(12) Patent Application Publication
(10) Pub. No.: US 2020/0172489 A1
(43) Pub. Date: Jun. 4, 2020

(54) FLUORESCENT COMPOUND AND AUTOPHAGY DETECTION REAGENT USING SAME

(71) Applicant: DOJINDO LABORATORIES, Kamimashiki-gun, Kumamoto (JP)

(72) Inventors: Hidefumi IWASHITA, Kamimashiki-gun, Kumamoto (JP); Ryo SAKAMOTO, Kamimashiki-gun, Kumamoto (JP); Takatoshi EZOE, Kamimashiki-gun, Kumamoto (JP); Nobuyuki OZEKI, Kamimashiki-gun, Kumamoto (JP); Munetaka ISHIYAMA, Kamimashiki-gun, Kumamoto (JP)

(73) Assignee: DOJINDO LABORATORIES, Kamimashiki-gun, Kumamoto (JP)

(21) Appl. No.: 16/613,200

(22) PCT Filed: May 18, 2018
(86) PCT No.: PCT/JP2018/019418

§ 371 Date: Nov. 13, 2019

(30) Foreign Application Priority Data
May 19, 2017 (JP) 2017-100197

(51) Int. Cl. C07D 221/14; C07D 401/10; A61K 49/00

(52) U.S. Cl. CPC C07D 221/14; A61K 49/0019; C07D 401/10

(57) ABSTRACT

A fluorescent compound or a salt thereof represented by General Formulae (I) or (II) shown below:

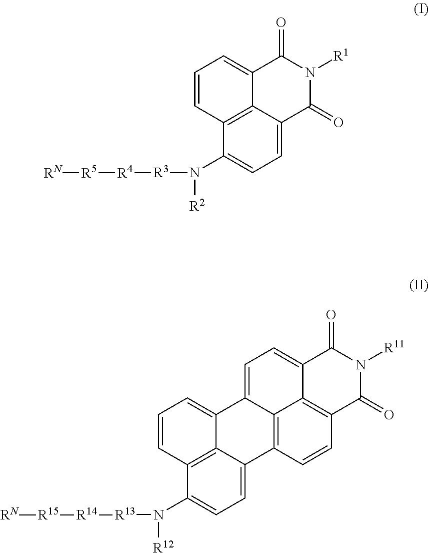

In the General Formulae (1) and (II) shown above, R¹ and R¹¹ represent an alkyl group or an ω-aminoalkyl group, R² and R¹² represent a hydrogen atom or a alkyl group, R³ and R¹³ represent an atomic group represented by a formula —(CH₂)ₘ— (m is a natural number of 10 or less), R⁴ and R¹⁴ represent an atomic group represented by a formula —CH₂— or —NR⁶— (—NR¹⁶—) (R⁶ and R¹⁶ represent an alkyl group), R⁵ and R¹⁵ represent an atomic group represented by a formula —(CH₂)ₙ— (n is a natural number of or less), R represents an atomic group represented by any one of formulae —NH₂, —NHR⁷—, NR⁷R⁸ and —NR⁷R⁸R⁹ (—NHR¹⁷, —NR¹⁷R¹⁸ and —N⁺R¹⁷R¹⁸R¹⁹) (R⁷, R⁸, R⁹, R¹⁷, R¹⁸ and R¹⁹ independently represents an alkyl group, respectively) and when R² and R¹² are alkyl groups and R⁴ (R¹⁴) is an atomic group represented by the formula —NR⁶—(—NR¹⁶—), R² and R⁶ and R¹² and R¹⁶ may bind with each other to form a ring.

### TECHNICAL FIELD

The present invention relates to a novel fluorescent compound and an autophagy detection reagent using same.

### BACKGROUND ART

In eukaryote from yeast to human, a decomposition process for recycling or metabolizing unnecessary cellular component such as protein and organelle called autophagy exists universally. Autophagy has been considered to be a mechanism for surviving a starved state by decomposing oneself non-selectively for securing the nutrition in a nutrition-starved state, however, later study revealed that it involves maintenance of homeostasis, programmed cell death in ontogenic process, suppression of diseases such as Huntington's disease, suppression of canceration of cells through removing denatured protein, preventing accumulation of excess mount of protein produced in the cells, removing deteriorated organelle or pathogenic microorganisms.

It is known that autophagy has a plurality of processes with different mechanisms such as macro autophagy, micro autophagy, and chaperone-mediated autophagy. Early research using an electron microscope revealed that, in macro autophagy, a separation membrane composed of bilayer membrane gradually extends to encapsulate degradation substrates such as unnecessary substances to form autophagosomes, and then the content of the autophagosome is decomposed by digestive enzyme in autolysosomes formed by fusing the autophagosome and lysosomes. Any processes are common in that the decomposition substrate is migrated into lysosome, where it is subjected to decompose.

In recent years, molecular mechanism of the autophagy has been elucidated and genes associated with autophagy have been identified. As a method for detecting autophagy, a method in which cells are lysed and the expression level of factors associated with autophagy from mRNA obtained is determined by a Western Blot method or immunostaining method is known (for example, see Patent Literature 1). However, this method cannot be applied to a cell live imaging since cells are lysed in this method.

As methods for detecting autophagy in living cells, introducing a plasmid vector coding LC3-GFP in which GFP is incorporated into LC3, a kind of gene product involving the formation of the autophagosomes (Atg protein) into cells and monitoring the expression of LC3 by a luminescence of GFP (for example, see Non-Patent Literature 1). Since the fluorescent intensity of GFP decreases under acidic condition, it is superior for detecting the early stage of autophagy. However, this method cannot be applied to any kind of cells since it requires expression of LC3-GFP in the cells.

A typical intracellular imaging method includes a method in which an intensity of fluorescence from a pH responsive fluorescent protein called Keima expressed in the cells (for example, see Non-Patent Literature 2) is monitored. The excitation spectrum of Keima varies in accordance with pH. A short wavelength (440 nm) peak is predominant in a neutral environment, whereas a long wavelength (550 nm) peak is predominant in an acidic environment. In a ratio (550 nm/440 nm) image obtained from two images measured using these two different excitation wavelengths, Keima in the neutral environment shows lower ratio value, whereas Keima in the acidic environment shows higher ratio value. Using this phenomenon, each step of autophagy (formation of autophagosome, fusion with lysosomes etc.) may be detected by reading out the pH change of the decomposition substrate associated with autophagy from the fluorescence image. However, this method is not applicable to all kinds of cells because the expression of Keima in the cells is required.

On the other hand, a method using monodansyl cadaverin (MDC) is known as an example of a method for detecting autophagy using low molecular fluorescent dye (for example, see Non-Patent Literature 3). However, the excitation wavelength of MDC is in an ultraviolet region, which may cause problems of cytotoxicity and breaching. Recently, CYTO-ID (Trademark) has been developed by Enzo Life Sciences, Inc. as a novel dye by which the problems of MDC have been solved (see Non-Patent Literature 4).

### CITATION LIST

### Patent Literatures

Patent Literature 1: Unexamined Japanese Patent Application Kokai Publication No. 2013-99305 (paragraph 0016).

### Non-Patent Literatures

Non-Patent Literature 1: Kuma A et al. The role of autophagy during the early neonatal starvation period. Nature Vol. 432, 1032-1036 (2004).
Non-Patent Literature 2: T. Kogure, et al. A Fluorescent variant of a protein from the stony coral Montipora facilitates dual-color single-laser fluorescence cross-correlation spectroscopy; Nature Biotechnology, vol. 24, 577-581 (2006).
Non-Patent Literature 3: “Autophagy Preceded Apoptosis in Oridonin-Treated Human Breast Cancer MCF-7 Cells”, Qiao CUI et al., Biol. Pharm. Bull., vol. 3, No. 5, p. 859-864 (2007).
Non-Patent Literature 4: http:/wwww.enzolifesciences.com/ENZ-51031/cyto-id-autophagy-detection-kit/

### SUMMARY OF INVENTION

### Problem to be Solved by Invention

However, which step of autophagy is detected is not clear by using the dye described in the Non-Patent Literature 4 since it has no pH-responsibility.

The present disclosure is achieved under such circumstances and the object of the present disclosure is to provide a fluorescent compound by which autophagy may be detected in all kinds of cells without a complicated process such as gene-recombination and an autophagy detection reagent using same.

### Means to Solve the Problem

First aspect of the invention along with the aforementioned object solves the problem as mentioned above by providing a fluorescent compound represented by General Formula (I) shown below.

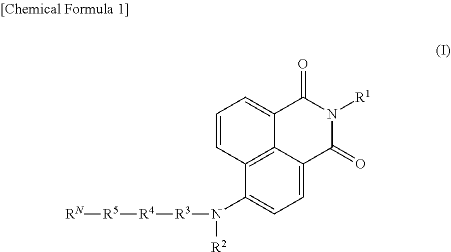

In the General Formula (I) shown above,

R¹ represents an alkyl group or ω-aminoalkyl group,

R² represents a hydrogen atom or an alkyl group,

R³ represents an atomic group represented by a formula —(CH₂)ₘ— (m is a natural number of 10 or less),

R⁴ represents an atomic group represented by a formula —CH₂— or —NR⁶— (R⁶ represents an alkyl group),

R⁵ represents an atomic group represented by a formula —(CH₂)ₙ— (n is a natural number of 10 or less),

R^{N} is an atomic group represented by any one of formulae —NH₂, —NHR⁷, —NR⁷R⁸ and —N⁺R⁷R⁸R⁹ (R⁷, R⁸ and R⁹ independently represent an alkyl group, respectively),

when R² is the alkyl group and R⁴ is the atomic group represented by the formula —NR⁶—, R² and R⁶ may bind with each other to form a ring.

The fluorescent compound according to the first aspect of the present disclosure may be represented by any one of Formulae 4a to 4f, 6h and 6i or a salt thereof.

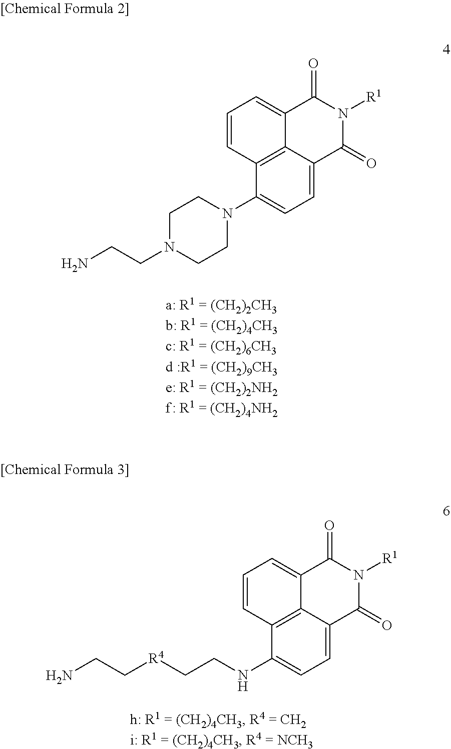

The fluorescent compound according to the first aspect of the present disclosure is preferably a compound represent by Formula 4b and 6h or a salt thereof.

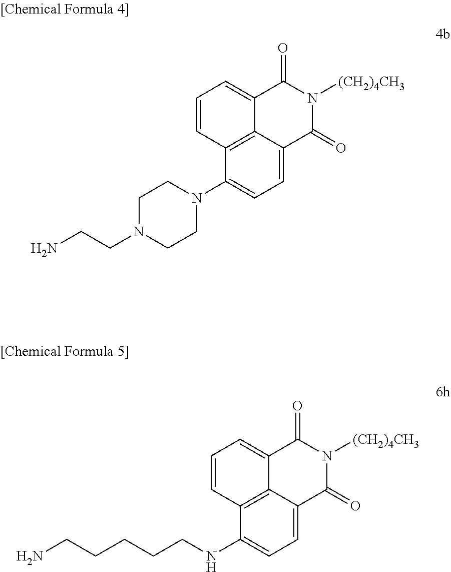

Second aspect of the invention solves the problem as mentioned above by providing a fluorescent compound represented by General Formula (II) shown below.

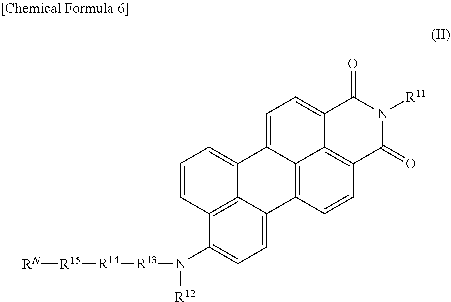

In the General Formula (II) shown above,

R¹¹ represents an alkyl group or an ω-aminoalkyl group,

R¹² represents a hydrogen atom or an alkyl group,

R¹³ represents an atomic group represented by a formula —(CH₂)ₘ— (m is a natural number of 10 or less),

R¹⁴ represents an atomic group represented by a formula —CH₂— or —NR¹⁶⁻(R¹⁶ represents an alkyl group),

R¹⁵ represents an atomic group represented by a formula —(CH₂)— (n is a natural number of 10 or less),

R^{N} is an atomic group represented by any one of formulae —NH₂, —NHR¹⁷, —NR¹⁷R¹⁸ and —N⁺R¹⁷R¹⁸R¹⁹ (R¹⁷, R¹⁸ and R¹⁹ independently represents an alkyl group, respectively),

when R¹² is the alkyl group and R¹⁴ is the atomic group represented by the formula —NR¹⁶—, R¹² and R¹⁶ may bind with each other to form a ring.

The fluorescent compound according to the second aspect of the present disclosure is preferably a compound represent by Formula 11 and 13 or a salt thereof.

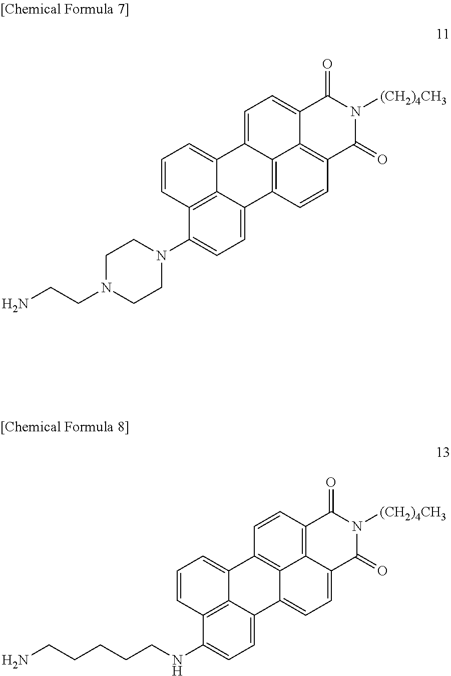

Third aspect of the invention solves the problem as mentioned above by providing an autophagy detection reagent comprising one or more selected from the group consisting of the fluorescent compounds and the salts thereof according to the first or second aspect of the present disclosure.

The autophagy detection reagent according to the third aspect of the present disclosure may comprise one or more selected from a group consisting of fluorescent compounds and salts thereof represented by the Formulae 4a to 4f shown above and one or more selected from a group consisting of fluorescent compound represented by the Formulae 6h and 6i shown above and salts thereof.

The autophagy detection reagent according to the third aspect of the present disclosure preferably comprises a fluorescent compound represented by the Formula 4b shown above or a salt thereof and a fluorescent compound represented by the Formula 6h shown above or a salt thereof.

The autophagy detection reagent according to the third aspect of the present disclosure may comprise a fluorescent compound represented by Formula 11 shown below or a salt thereof and a fluorescent compound represented by Formula 13 shown below or a salt thereof.

The autophagy detection reagent according to the third aspect of the present disclosure preferably comprises one or more selected from a group consisting of fluorescent compounds represented by General Formula (Ia) shown below and a salt thereof and one or more selected from a group consisting of fluorescent compounds represented by General Formula (IIb) and a salt thereof; or one or more selected from a group consisting of fluorescent compounds represented by General Formula (Ib) shown below and a salt thereof and one or more selected from a group consisting of fluorescent compounds represented by General Formula (Ha) and a salt thereof.

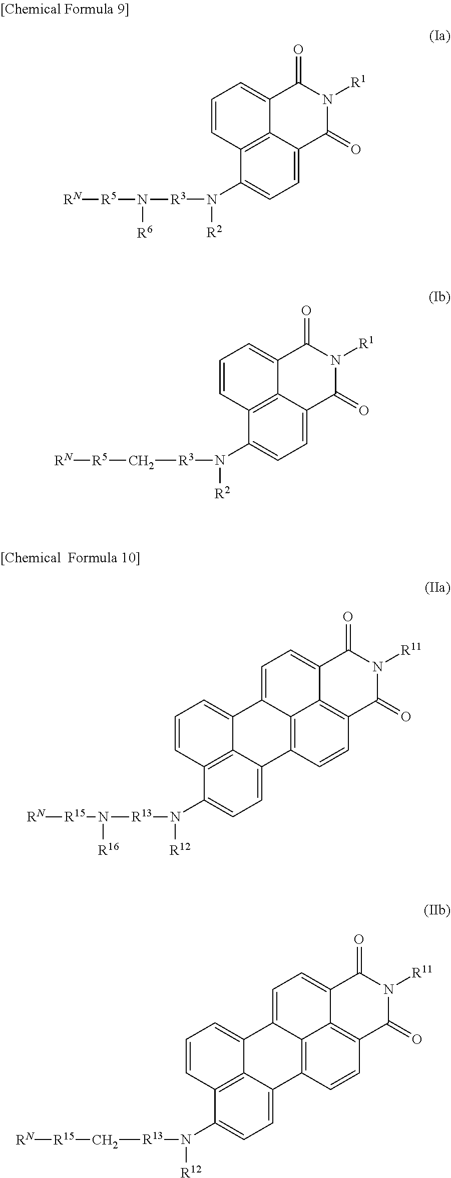

In the General Formulae (Ia) and (Ib) shown above,

R¹ represents an alkyl group or ω-aminoalkyl group,

R² represents a hydrogen atom or an alkyl group,

R³ represents an atomic group represented by a formula (CH₂)ₘ— (m is a natural number of 10 or less),

R⁵ represents an atomic group represented by a formula —(CH₂)ₙ— (n is a natural number of 10 or less), R^{N} is an atomic group represented by any one of formulae —NH₂, —NHR⁷, —NR⁷R⁸ and —N⁺R⁷R⁸R⁹ (R⁷, R⁸ and R⁹ independently represent an alkyl group, respectively),

in the General Formula (Ia) shown above, R⁶ represents an alkyl group, R² and R⁶ may bind with each other to form a ring,

in the General Formula (IIa) and (IIb) shown above,

R¹¹ represents an alkyl group or an ω-aminoalkyl group,

R¹² represents a hydrogen atom or an alkyl group,

R¹³ represents an atomic group represented by a formula —(CH₂)ₘ— (m is a natural number of 10 or less),

R¹⁵ represents an atomic group represented by a formula —(CH₂)ₙ— (n is a natural number of 10 or less),

R^{N} is an atomic group represented by any one of formulae —NH₂, —NHR¹⁷, —NR¹⁷R¹⁸ and —N⁺R¹⁷R¹⁸R¹⁹ (R¹⁷, R¹⁸ and R¹⁹ independently represents an alkyl group, respectively), in the General Formula (IIa), R¹⁶ represents an alkyl group, R¹² and R¹⁶ may bind with each other to form a ring.

The autophagy detection reagent according to the third aspect of the present disclosure preferably comprises a fluorescent compound represented by the Formula 6h shown below or a salt thereof and a fluorescent compound represented by the Formula 11 shown above or a salt thereof; or a fluorescent compound represented by the Formula 4b shown above or a salt thereof and a fluorescent compound represented by the Formula 13 shown above or a salt thereof.

### Advantageous Effect of the Invention

In the fluorescent compound represented by General Formulae shown above, naphthalimide and perylene imide which emit a fluorescence in a hydrophobic field as a fluorescent chromophore group. Therefore, as the fluorescent intensity increases by incorporation into autophagosomes or autolysosomes, autophagy may read out by the fluorescent emission. Also, regulation of hydrophobicity and impartion of pH responsivity of fluorescent intensity using photo induced electron transfer (PET) or fluorescent wavelength may be easily performed by selecting functional groups R1 to R5 appropriately. In addition, combining a plurality of the fluorescent compounds having different sensitivities to internal conditions of autophagosomes and autolysosomes in each step of autophagy (such as pH) enables the observation of each step of autophagy. Particularly, in addition to the sensitivities to internal conditions of autophagosomes and autolysosomes, using a combination of one or more selected from a group consisting of fluorescent compounds represented by General Formula (Ia) shown below and a salt thereof and one or more selected from a group consisting of fluorescent compounds represented by General Formula (IIb) and a salt thereof; or one or more selected from a group consisting of fluorescent compounds represented by General Formula (Ib) shown below and a salt thereof and one or more selected from a group consisting of fluorescent compounds represented by General Formula (IIa) and a salt thereof having different emission wavelength enables detecting each step of autophagy stepwise.

### BRIEF DESCRIPTION OF THE DRAWINGS

FIG. 1 shows a graph showing pH dependence of fluorescent intensities of the fluorescent Compounds 4a, 4b, 4e and 4f.

FIG. 2 shows fluorescent microscopic images of HeLa cells after introducing the Compound 4b and incubating for 6 hours and 20 hours.

FIG. 3 shows results of flow cytometric measurement of HeLa cells after introducing the Compound 4b and incubating for 6 hours and 20 hours under starvation condition.

FIG. 4 shows fluorescent microscopic images showing the changes of the fluorescent intensities in HeLa cells of which autophagy was induced by Rapamycin in the presence of chloroquine or starvation induction.

FIG. 5 shows photographic images showing the result of the quantitative expression amount of LC3 in HeLa cells of which autophagy was induced by Rapamycin or starvation induction in the presence of chloroquine.

FIG. 6 shows a graph showing pH dependence of fluorescent intensities of the fluorescent Compounds 4b and 6h.

FIG. 7 shows fluorescent microscopic images showing the result of the comparison of the fluorescent intensities of Compound 4b and Compound 6h introduced in HeLa cells.

FIG. 8 shows high-magnification fluorescent microscopic images showing the result of the comparison of the fluorescent intensities of Compound 4b and Compound 6h introduced in HeLa cells.

FIG. 9 shows a graph showing pH dependence of fluorescent intensities of the fluorescent Compound 11.

FIG. 10 shows fluorescent microscopic images of HeLa cells after introducing the Compound 11 and incubating for 5 hours.

FIG. 11 shows fluorescent microscopic images of HeLa cells after introducing the Compound 6h and Compound 11 and incubating for 3 hours and 6 hours.

FIG. 12 shows fluorescent microscopic images of HeLa cells after introducing the Compound 4b and Compound 13 and incubating for 3 hours and 6 hours.

### EMBODIMENTS OF INVENTION

A fluorescent compound according to first embodiment of the present disclosure is represented by General Formula (I).

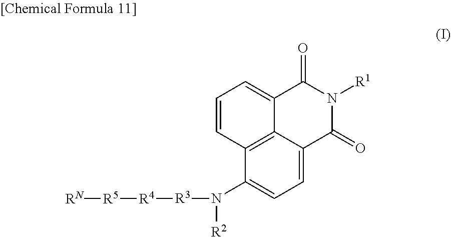

In the General Formula (I) shown above, R¹ represents an alkyl group or an ω-aminoalkyl group. Although the alkyl group or ω-aminoalkyl group may have a branch or substituent, a linear alkyl group or ω-aminoalkyl group is preferred, of which carbon number is not particularly limited, it is preferably 1 to 18, more preferably 1 to 12, particularly preferably 1 to 10.

In the General Formula (I) shown above, R² represents a hydrogen atom or an alkyl group. Although the alkyl group may have a branch or substituent, a linear alkyl group is preferred, of which carbon number is not particularly limited, it is preferably 1 to 18, more preferably 1 to 12, particularly preferably 1 to 10.

In the General Formula (I) shown above, R³ represents an atomic group represented by a formula —(CH₂)ₘ—, m is a natural number of 10 or less, preferably 2 to 6.

In the General Formula (I) shown above, R⁴ represents an atomic group represented by a formula —CH₂— or —NR⁶—, R⁶ represents an alkyl group. Although the alkyl group may have a branch or substituent, a linear alkyl group is preferred, of which carbon number is not particularly limited, it is preferably 1 to 18, more preferably 1 to 12, particularly preferably 1 to 10.

In the General Formula (I) shown above, R⁵ represents an atomic group represented by a formula —(CH₂)ₙ—, n is a natural number of 10 or less, preferably 2 to 6.

In the General Formula (I) shown above, R^{N} represents an atomic group represented by any one of the formulae —NH₂, —NHR⁷, —NR⁷R⁸ and —N⁺R⁷R⁸R⁹, R⁷, R⁸ and R⁹ independently represents an alkyl group, respectively. Although the alkyl group may have a branch or substituent, a linear alkyl group is preferred, of which carbon number is not particularly limited, it is preferably 1 to 18, more preferably 1 to 12, particularly preferably 1 to 10.

When R^{N} is the atomic group represented by the formula —NH₂, —NHR⁷ or —NR⁷R⁸, R^{N} may form a salt of which nitrogen atom is protonated. Kind of the salt is not particularly limited as long as it does not affect the fluorescent intensity, particular example of which includes hydrochloride salt, hydrobromide salt, nitrate salt, sulfate salt, hydrogen sulfate salt, carbonate salt, hydrogen carbonate salt, phosphate salt, hydrogen phosphate salt, dihydrogen phosphate salt, acetate salt, propionate salt, lactate salt, tartrate salt, citrate salt, methanesulfonate salt and benzenesulfonate salt. When R^{N} is the atomic group represented by the formula —N⁺R⁷R⁸R⁹, R^{N} also may form a salt similar to that described above.

In the General Formula (I) shown above, when R² is the alkyl group and R⁴ is the atomic group represented by the formula —NR⁶—, R² and R⁶ may bind to each other to form a ring containing nitrogen atom(s) such as piperazine ring.

Preferred example of the fluorescent compound represented by the General Formula (I) includes the compound represented by any one of the formulae 4a to 4f, 6h and 6i and a salt thereof.

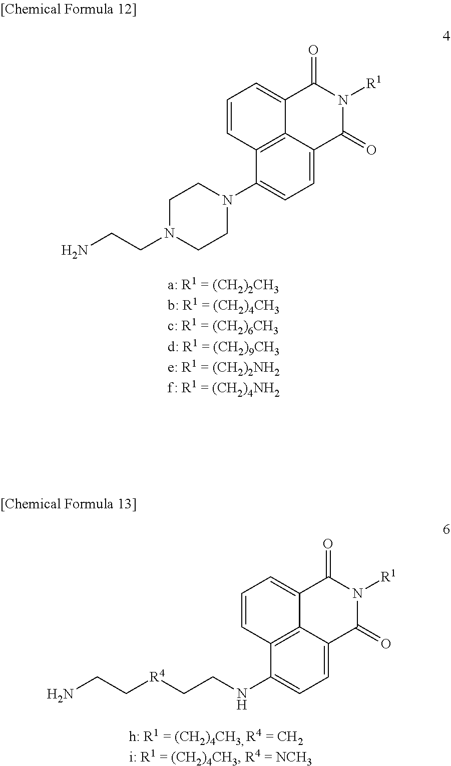

These compounds have a naphtalimide group which emits fluorescence in a hydrophobic field, which is designed to emit the fluorescence only in the case that it is incorporated into the autophagosomes. In addition, in the fluorescent compounds represented by the formulae 4a to 4f and 6i, the fluorescence is quenched by a photo-induced electron transfer (PET) from a non-covalent electron pair, whereas under the acidic condition, when the nitrogen atom is protonated, the fluorescent intensity increases. Therefore, these fluorescence compounds may be preferably applied to read out the step later than the fusion with the lysosomes in the autophagy. On the other hand, since the fluorescent intensity of the fluorescent compound represented by the formula 6h which has no nitrogen atom on the side chain is not affected by pH, it may be preferably applied to the read out of earlier step of the autophagy. Furthermore, all steps of the autophagy may be read out by the change of the fluorescent intensity by combining both compounds appropriately.

The fluorescent compound represented by the General Formula (I) may be synthesized by using the method known in the art. For example, the compound represented by the formula 4a to 4f, 6h and 6i (their hydrochloride salts) may be synthesized according to a scheme shown below.

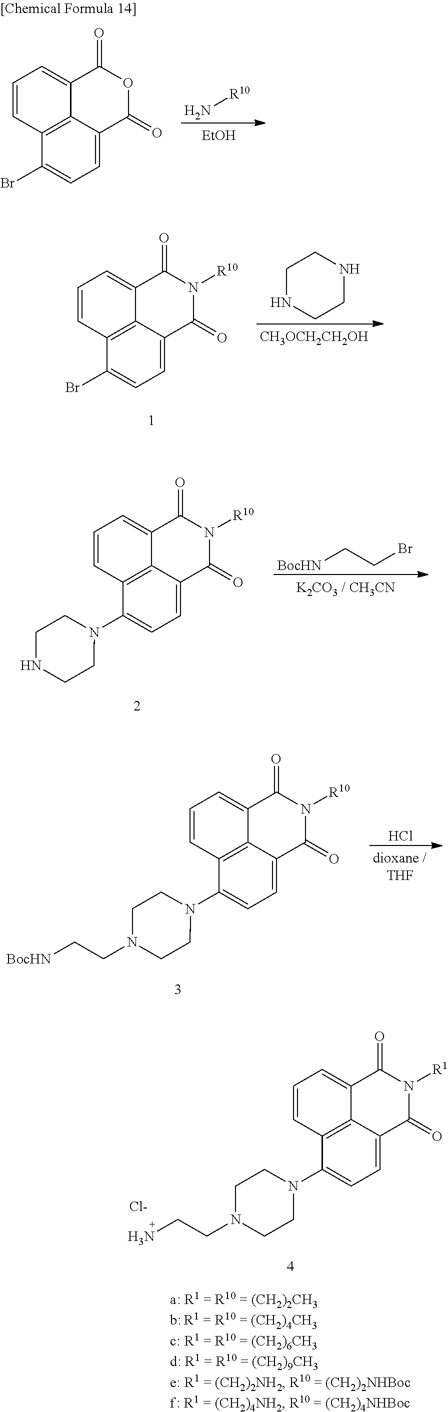

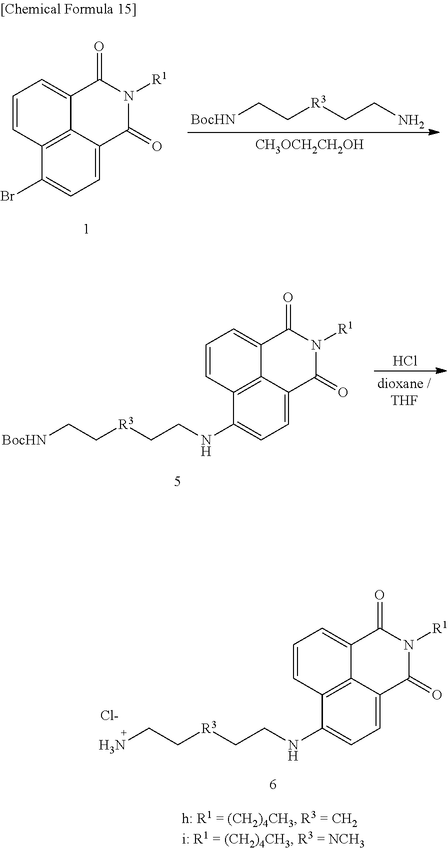

A fluorescent compound according one embodiment of the present disclosure is represented by General Formula (II).

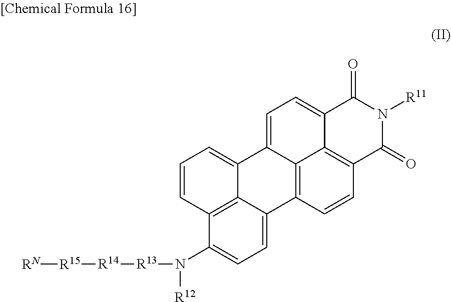

In the General Formula (II) shown above, R¹¹ represents an alkyl group or an ω-aminoalkyl group. Although the alkyl group or ω-aminoalkyl group may have a branch or substituent, a linear alkyl group or ω-aminoalkyl group is preferred, of which carbon number is not particularly limited, it is preferably 1 to 18, more preferably 1 to 12, particularly preferably 1 to 10.

In the General Formula (II) shown above, R¹² represents a hydrogen atom or an alkyl group. Although the alkyl group may have a branch or substituent, a linear alkyl group is preferred, of which carbon number is not particularly limited, it is preferably 1 to 18, more preferably 1 to 12, particularly preferably 1 to 10.

In the General Formula (II) shown above, R¹³ represents an atomic group represented by a formula —(CH₂)ₘ—, m is a natural number of 10 or less, preferably 2 to 6.

In the General Formula (II) shown above, R¹⁴ represents an atomic group represented by a formula —CH₂— or —NR¹⁶—, R¹⁶ represents an alkyl group. Although the alkyl group may have a branch or substituent, a linear alkyl group is preferred, of which carbon number is not particularly limited, it is preferably 1 to 18, more preferably 1 to 12, particularly preferably 1 to 10.

In the General Formula (II) shown above, R¹⁵ represents an atomic group represented by a formula —(CH₂)ₙ—, n is a natural number of 10 or less, preferably 2 to 6.

In the General Formula (II) shown above, R^{N} represents an atomic group represented by any one of the formulae —NH₂, —NHR¹⁷, —NR¹⁷R¹⁸ and —N⁺R¹⁷R¹⁸R¹⁹, R¹⁷, R¹⁸ and R¹⁹ independently represents an alkyl group, respectively. Although the alkyl group may have a branch or substituent, a linear alkyl group is preferred, of which carbon number is not particularly limited, it is preferably 1 to 18, more preferably 1 to 12, particularly preferably 1 to 10.

When R^{N} is the atomic group represented by the formula —NH₂, —NHR¹⁷ or —NR¹⁷R¹⁸, R^{N} may form a salt of which nitrogen atom is protonated. Kind of the salt is not particularly limited as long as it does not affect the fluorescent intensity, particular example of which includes hydrochloride salt, hydrobromide salt, nitrate salt, sulfate salt, hydrogen sulfate salt, carbonate salt, hydrogen carbonate salt, phosphate salt, hydrogen phosphate salt, dihydrogen phosphate salt, acetate salt, propionate salt, lactate salt, tartrate salt, citrate salt, methanesulfonate salt and benzenesulfonate salt. When R^{N} is the atomic group represented by the formula —N⁺R¹⁷R¹⁸R¹⁹, R^{N} also may form a salt similar to that described above.

In the General Formula (II) shown above, when R¹² is the alkyl group and R¹⁴ is the atomic group represented by the formula —NR¹⁶—, R¹² and R¹⁶ may bind to each other to form a ring containing nitrogen atom(s) such as piperazine ring.

Preferred example of the fluorescent compound represented by the General Formula (II) includes the compound represented by any one of the formulae 11 and 3 and a salt thereof.

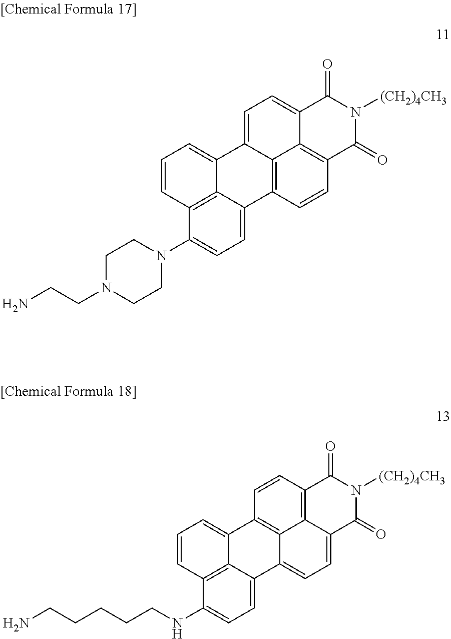

These compounds have a perylene imide group which emits fluorescence in a hydrophobic field, which is designed to emit the fluorescence only in the case that it is incorporated into the autophagosomes. In addition, in the fluorescent compound represented by the formula 11, the fluorescence is quenched by a photo-induced electron transfer (PET) from a non-covalent electron pair, whereas under the acidic condition, when the nitrogen atom is protonated, the fluorescent intensity increases. Therefore, these fluorescence compounds may be preferably applied to read out the step later than the fusion with the lysosomes in the autophagy.

The fluorescent compound represented by the General Formula (II) may be synthesized by using the method known in the art. For example, the compound represented by the formula 11 (its hydrochloride salts) may be synthesized according to a scheme shown below.

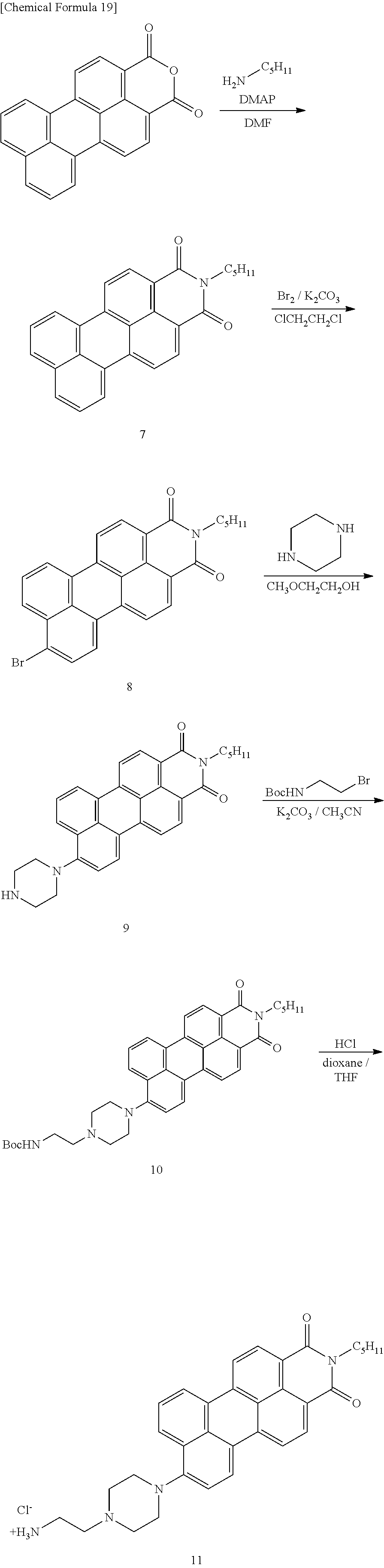

The compound represented by the formula 13 (its hydrochloride salts) may be synthesized according to a scheme shown below.

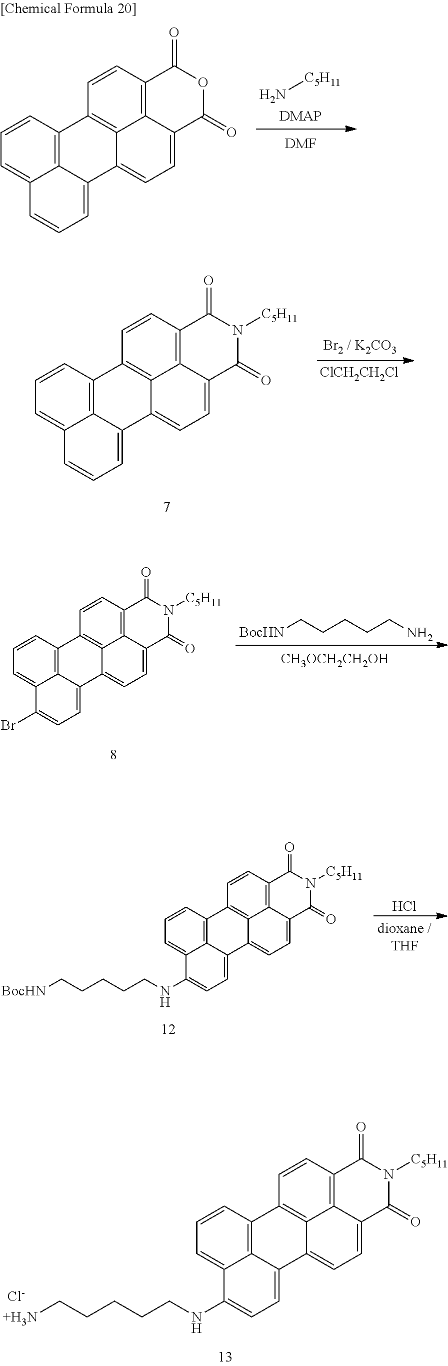

Since the compound represent by the General Formula (I) and (II) shown above (hereinafter it may be abbreviated to “the compound”) has permeability to cell membrane, autophagosomes and lysosomes (autolysosomes), introduction of the compound to cells may be carried out by simply contacting the compound to the cell without using special technique. Thus, the autophagy in cells may be detected by incubating the cells in which the compound has been introduced for certain period and measuring a fluorescent emission from the cells using any known means such as fluorescent microscopy. Certain embodiment of the present disclosure relates to a method for detecting autophagy comprising a step for administering the fluorescent compound represented by General Formulae shown above into cells and a step for measuring a fluorescent emission from the cells after incubating for certain period.

In the fluorescent compounds represented by the General Formulae (Ia) or (IIa), the fluorescence is quenched by a photo-induced electron transfer (PET) from a non-covalent electron pair, whereas under the acidic condition, when the nitrogen atom is protonated, the fluorescence intensity increases. Therefore, these fluorescence compounds may be preferably applied to read out the step later than the fusion with the lysosomes in the autophagy. On the other hand, since the fluorescent intensity of the fluorescent compound represented by the General Formulae (Ib) or (IIb) which has no nitrogen atom on the side chain is not affected by pH, it may be preferably applied to the read out of earlier step of the autophagy. Therefore, all steps of the autophagy may be read out by the change of the fluorescent wavelength and the fluorescent intensity by combining one or more selected from a group consisting of fluorescent compounds represented by General Formula (Ia) shown below and a salt thereof and one or more selected from a group consisting of fluorescent compounds represented by General Formula (IIb) and a salt thereof; or one or more selected from a group consisting of fluorescent compounds represented by General Formula (b) shown below and a salt thereof and one or more selected from a group consisting of fluorescent compounds represented by General Formula (IIa) and a salt thereof.

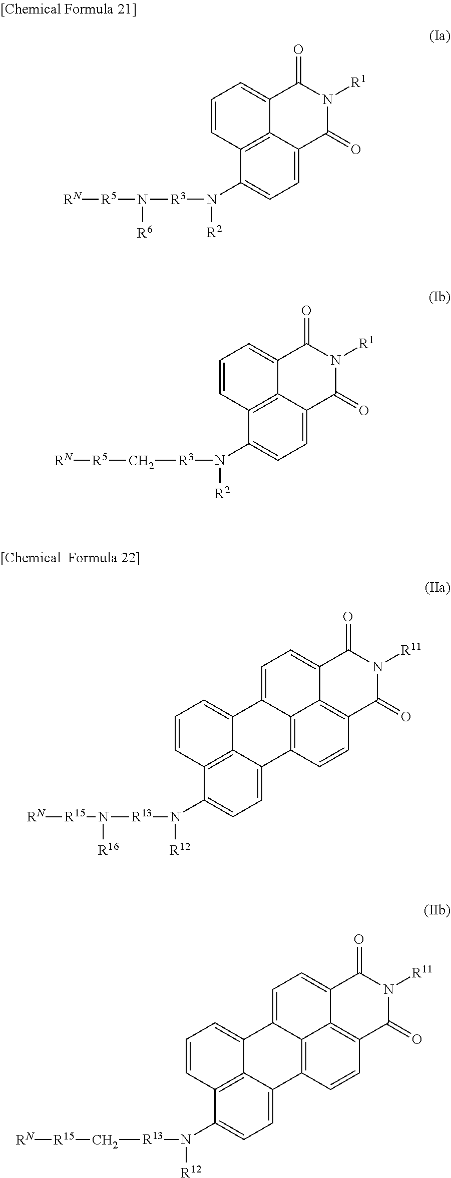

In the General Formulae (Ia) and (Ib) shown above,

R¹ represents an alkyl group or ω-aminoalkyl group,

R² represents a hydrogen atom or an alkyl group,

R³ represents an atomic group represented by a formula —(CH₂)ₘ— (m is a natural number of 10 or less),

R⁵ represents an atomic group represented by a formula —(CH₂)ₙ— (n is a natural number of 10 or less),

R^{N} is an atomic group represented by any one of formulae —NH₂, —NHR⁷, —NR⁷R⁸ and —NR⁷R⁸R⁹ (R⁷, R⁸ and R⁹ independently represent an alkyl group, respectively),

R² and R⁶ in the General Formula (Ia) may bind to each other to form a ring,

in the General Formula (IIa) and (IIb) shown above,

R¹¹ represents an alkyl group or an ω-aminoalkyl group,

R¹² represents a hydrogen atom or an alkyl group,

R¹³ represents an atomic group represented by a formula —(CH₂)ₘ— (m is a natural number of 10 or less),

R¹⁵ represents an atomic group represented by a formula —(CH₂)ₙ— (n is a natural number of 10 or less),

R^{N} is an atomic group represented by any one of formulae —NH₂, —NHR¹⁷, —NR¹⁷R¹⁸ and —N⁺R¹⁷R¹⁸R¹⁹ (R¹⁷, R¹⁸ and R¹⁹ independently represents an alkyl group, respectively),

in the General Formula (IIa), R¹⁶ represents an alkyl group, R¹² and R¹⁶ may bind with each other to form a ring.

An example of the preferred combination mentioned above includes the combination of the fluorescent compound represented by the formula 6h shown above or the salt thereof and the fluorescent compound represented by the formula 11 shown above or the salt thereof; or the combination of the fluorescent compound represented by the formula 4b shown above or the salt thereof and the fluorescent compound represented by the formula 13 shown above or the salt thereof.

The compound is used in the form of a solution or a dispersion in which the compound is dissolved or dispersed in an appropriate solvent of buffer in certain concentration to introduce into cells. Certain embodiment of the present disclosure relates to an autophagy detecting reagent in which the compound is dissolved or dispersed in an appropriate solvent of buffer in certain concentration.

### EXAMPLES

The present invention will be illustrated by referring the examples carried out to confirm the action and the effect of the present invention. In the Examples hereinafter, “the compound represented by the formula X” may be abbreviated to “the Compound X”.

### Example 1: Preparation of Fluorescent Compound (1)

### Synthesis of 6-bromo-2-propyl-1H-benzo[de]isoquinoline-1,3(2H)-dione (1a)

To a 200 mL eggplant-shaped flask, 4-bromo-1,8-naphthalic anhydride (1g, 3.6 mmol), propyl amine (298 mg, 1.4 eq., 5.04 mmol), DMAP (528 mg, 1.2 eq., 4.3 mmol) and 50 mL of ethanol were added and refluxed at 80° C. for 16 hours. After cooling to room temperature, crystal precipitated was filtrated to give 930 mg of yellow crystal (yield: 84%).

¹H-NMR (400 MHz, CDCl₃) δ: 8.65 (d, 1H, J=7.2 Hz), 8.55 (d, 1H, J=8.5 Hz), 8.40 (d, 1H, J=7.8 Hz), 8.03 (d, 1H, J=7.8 Hz), 7.84 (t, 1H, J=7.8 Hz), 4.13 (t, 2H, J=7.5 Hz), 1.79-1.71 (m, 2H), 1.01 (t, 3H, J=7.3 Hz); ¹³C-NMR (101 MHz, CDCl₃): δ 163.5, 133.1, 131.9, 131.1, 131.0, 130.5, 130.1, 128.9, 128.0, 123.1, 122.2, 42.0, 21.3, 11.5.

### Synthesis of 6-bromo-2-pentyl-1H-benzo[de]isoquinoline-1,3(2H)-dione (1b)

Synthesis was conducted similarly to that of the Compound 1a using 4-bromo-1,8-naphthalic anhydride (1 g, 3.6 mmol), amyl amine (436 mg, 1,4 eq., 5.04 mmol), DMAP (528 mg, 1.2 eq., 4.3 mmol) and 50 mL of ethanol to give 1.2 g of yellow crystal (yield: 80%).

¹H-NMR (400 MHz, CDCl₃) δ: 8.65 (d, 1H, J=7.2 Hz), 8.56 (d, 1H, J=8.5 Hz), 8.40 (d, 1H, J=7.8 Hz), 8.03 (d, 1H, J=7.8 Hz), 7.84 (t, 1H, J=7.8 Hz), 4.16 (t, 2H, J=7.5 Hz), 1.75-1.69 (m, 2H), 1.42-1.38 (m, 4H), 0.91 (t, 3H, J=7.3 Hz); ¹³C-NMR (101 MHz, CDCl₃): δ 163.5, 133.1, 131.9, 131.1, 131.0, 130.5, 130.1, 128.9, 128.0, 123.1, 122.3, 40.6, 29.2, 27.7, 22.4, 14.0.

### Synthesis of 6-bromo-2-heptyl-1H-benzo[de]isoquinoline-1,3(2H)-dione (1c)

Synthesis was conducted similarly to that of the Compound 1a using 4-bromo-1,8-naphthalic anhydride (2.0 g, 7.2 mmol), heptyl amine (1.1 g, 1,4 eq., 10.1 mmol), DMAP (1.0 g, 1.2 eq., 8.6 mmol) and 100 mL of ethanol to give 1.5 g of yellow crystal (yield: 55.6%).

¹H-NMR (400 MHz, CDCl₃) δ: 8.65 (d, 1H, J=7.2 Hz), 8.56 (d, 1H, J=8.5 Hz), 8.41 (d, 1H, J=7.8 Hz), 8.03 (d, 1H, J=7.8 Hz), 7.84 (t, 1H, J=7.8 Hz), 4.16 (t, 2H, J=7.5 Hz), 1.76-1.68 (m, 2H), 1.43-1.30 (m, 8H), 0.89 (t, 3H, J=7.3 Hz); ¹³C-NMR (101 MHz, CDCl₃): δ 163.5, 133.1, 131.9, 131.1, 131.0, 130.5, 130.1, 128.9, 128.0, 123.1, 122.2, 40.6, 31.7, 29.0, 28.0, 27.0, 22.6, 14.0.

### Synthesis of 6-bromo-2-decyl-1H-benzo[de]isoquinoline-1,3(2H)-dione (1d)

Synthesis was conducted similarly to that of the Compound 1a using 4-bromo-1,8-naphthalic anhydride (2.0 g, 7.2 mmol), 1-aminodecane (1.6 g, 1,4 eq., 10.1 mmol), DMAP (1.0 g, 1.2 eq., 8.6 mmol) and 100 mL of ethanol to give 1.5 g of yellow crystal (yield: 50%).

¹H-NMR (400 MHz, CDCl₃) δ: 8.65 (d, 1H, J=7.2 Hz), 8.56 (d, 1H, J=8.5 Hz), 8.41 (d, 1H, J=7.8 Hz), 8.04 (d, 1H, J=7.8 Hz), 7.84 (t, 1H, J=7.8 Hz), 4.16 (t, 2H, J=7.5 Hz), 1.76-1.68 (m, 2H), 1.45-1.25 (m, 17H), 0.88 (t, 3H, J=7.3 Hz); ¹³C-NMR (101 MHz, CDCl₃): δ 163.5, 133.1, 131.9, 131.1, 131.0, 130.5, 130.1, 128.9, 128.0, 123.1, 122.2, 40.6, 31.9, 29.5, 29.3, 28.0, 27.1 22.6, 14.1.

### Synthesis of tert-butyl (2-(6-bromo-1,3-dioxo-1H-benzo[de]isoquinoline-2(3H)-yl)ethyl) carbamate (1e)

Synthesis was conducted similarly to that of the Compound 1a using 4-bromo-1,8-naphthalic anhydride (1.0 g, 3.6 mmol), N-(tert-butoxycarbonyl)-1,2-diaminoethane (807 mg, 1,4 eq., 5.04 mmol), DMAP (528 mg, 1.2 eq., 4.3 mmol) and 50 mL of ethanol to give 1.3 g of yellow crystal (yield: 86%).

¹H-NMR (400 MHz, CDCl₃) δ: 8.66 (d, 1H, J=7.2 Hz), 8.57 (d, 1H, J=8.5 Hz), 8.41 (d, 1H, J=7.8 Hz), 8.04 (d, 1H, J=7.8 Hz), 7.84 (t, 1H, J=7.8 Hz), 4.93 (s, 1H), 4.35 (t, 2H, J=7.5 Hz), 3.54-3.53 (m, 2H), 1.27 (s, 9H, J=7.3 Hz); ¹³C-NMR (101 MHz, CDCl₃): δ 163.9, 156.0, 133.3, 132.2, 131.3, 131.0, 130.5, 130.3, 129.0, 128.0, 122.8, 122.0, 79.1, 40.0, 39.5, 28.2.

### Synthesis of tert-butyl (4-(6-bromo-1,3-dioxo-1H-benzo[de]isoquinoline-2(3H)-yl)butyl) carbamate (1f)

Synthesis was conducted similarly to that of the Compound 1a using 4-bromo-1,8-naphthalic anhydride (1.0 g, 3.6 mmol), N-(tert-butoxycarbonyl)-1,4-diaminobutane (948 mg, 1.4 eq., 5.04 mmol). DMAP (528 mg, 1.2 eq., 4.3 mmol) and 50 mL of ethanol to give 900 mg of yellow crystal (yield: 56%).

¹H-NMR (400 MHz, CDCl₃) δ: 8.65 (d, 1H, J=7.2 Hz), 8.57 (d, 1H, J=8.5 Hz), 8.41 (d, 1H, J=7.8 Hz), 8.04 (d, 1H, J=7.8 Hz), 7.85 (t, 1H, J=7.8 Hz), 4.62 (s, 1H), 4.18 (t, 2H, J=7.5 Hz), 3.20-3.18 (m, 2H), 1.79-1.75 (m, 2H), 1.62-1.57 (m, 2H), 1.42 (s, 9H, J=7.3 Hz); ¹³C-NMR (101 MHz, CDCl₃): δ 163.5, 155.9, 133.2, 132.0, 131.2, 131.0, 130.5, 130.2, 128.9, 128.0, 123.0, 122.1, 79.0, 40.2, 40.0, 28.4, 27.5, 25.4.

### Synthesis of 6-(piperazine-1-yl)-2-propyl-1H-benzo[de]isoquinoline-1,3(2H)-dione (2a)

To 200 mL of eggplant-shaped flask, Compound 1a (500 mg, 1.5 mmol), piperazine (1.3 g, 10 eq., 15 mmol) and 50 mL of 2-methoxyethanol were added and refluxed at 120° C. for 16 hours. The progress of the reaction was checked and the reaction solution was evaporated using an evaporator. A silica gel column was used for purification using a gradient from 100% chloroform to chloroform/methanol=7/3, 400 mg of yellow crystal was obtained (yield: 82%).

¹H-NMR (400 MHz, CDCl₃) δ: 8.58 (d, 1H, J=7.2 Hz), 8.52 (d, 1H, J=8.5 Hz), 8.42 (d, 1H, J=7.8 Hz), 7.69 (t, 1H, J=7.8 Hz), 7.21 (d, 1H, J=7.8 Hz), 4.13 (t, 2H, J=7.5 Hz), 3.22 (d, 8H, J=7.8 Hz), 1.78-1.73 (m, 2H), 1.01 (t, 3H, J=7.3 Hz); ¹³C-NMR (101 MHz, CDCl₃): δ 164.5, 164.0, 156.3, 132.5, 131.0, 130.2, 129.9, 126.2, 125.6, 123.3, 116.7, 114.9, 54.4, 46.2, 41.7, 21.4, 11.5.

### Synthesis of 2-pentyl-6-(piperazine-1-yl)-1H-benzo[de]isoquinoline-1,3(2H)-dione (2b)

The synthesis was conducted similarly to that of the compound 2a using Compound 1b (1g, 2.8 mmol), piperazine (2.6 g, 10 eq., 31 mmol) and 100 mL of 2-methoxyethanol to give 800 mg of yellow crystal (yield: 79%).

¹H-NMR (400 MHz, CDCl₃) δ: 8.56 (d, 1H, J=8.5 Hz), 8.51 (d, 1H, J=7.8 Hz), 8.41 (d, 1H, J=7.8 Hz), 7.69 (t, 1H, J=7.8 Hz), 7.21 (d, 1H, J=7.2 Hz), 4.15 (t, 2H. J=7.5 Hz), 3.24-3.21 (m, 2H), 1.91 (s, 2H), 1.72 (m, 2H), 1.39 (s, 4H), 0.91 (t, 3H, J=7.3 Hz); ¹³C-NMR (101 MHz, CDCl₃): δ 164.4, 163.9, 156.3, 132.5, 131.0, 130.2, 129.8, 126.1, 125.5, 123.2, 116.7, 114.8, 54.4, 46.2, 40.2, 29.2, 27.8, 22.4, 14.0.

### Synthesis of 2heptyl-6-(piperazine-1-yl)-1H-benzo[de]isoquinoline-1,3(2H)-dione (2c)

The synthesis was conducted similarly to that of the compound 2a using Compound 1c (1g, 2.6 mmol), piperazine (2.3 g, 10 eq., 26 mmol) and 100 mL of 2-methoxyethanol to give 800 mg of yellow crystal (yield: 79%).

¹H-NMR (400 MHz, CDCl₃) δ: 8.57 (d, 1H, J=8.5 Hz), 8.51 (d, 1H, J=7.8 Hz), 8.41 (d, 1H, J=7.8 Hz), 7.69 (t, 1H, J=7.8 Hz), 7.21 (d, 1H, J=7.2 Hz), 4.15 (t, 2H, J=7.5 Hz), 3.25-3.20 (m, 8H), 1.75-1.68 (m, 2H), 1.43-1.28 (m, 8H), 0.89 (t, 3H, J=7.3 Hz); ¹³C-NMR (101 MHz, CDCl₃): δ 164.3, 163.8, 156.2, 132.4, 130.9, 130.1, 129.7, 126.3, 126.0, 125.5, 123.2, 116.6, 114.8, 54.3, 46.2, 40.2, 31.7, 31.5, 29.0, 28.8, 28.1, 27.2, 27.1, 22.7, 22.5, 22.4, 14.0.

### Synthesis of 2decyl-6-(piperazine-1-yl)-1H-benzo[de]isoquinoline-1,3(2H)-dione (2d)

The synthesis was conducted similarly to that of the compound 2a using Compound 1d (1g, 2.4 mmol), piperazine (2.0 g, 10 eq., 24 mmol) and 100 mL of 2-methoxyethanol to give 800 mg of yellow crystal (yield: 79%).

¹H-NMR (400 MHz, CDCl₃) δ: 8.57 (d, 1H, J=8.5 Hz), 8.51 (d, 1H, J=7.8 Hz), 8.41 (d, 1H, J=7.8 Hz), 7.69 (t, 1H. J=7.8 Hz), 7.22 (d, 1H, J=7.2 Hz), 4.15 (t, 2H, J=7.5 Hz), 3.25-3.20 (m, 8H), 1.75-1.67 (m, 2H), 1.44-1.25 (m, 14H), 0.88 (t, 3H, J=7.3 Hz); ¹³C-NMR (101 MHz, CDCl₃): δ 164.3, 163.9, 156.2, 132.4, 130.9, 130.1, 129.8, 126.1, 125.8, 125.5, 123.2, 116.7, 114.8, 54.3, 46.2, 40.3, 31.8, 31.6, 29.5, 29.3, 29.2, 28.1, 27.1, 22.6, 22.4, 14.1.

### Synthesis of tert-butyl (2-(1,3-dioxo-6-(piperazine-1-yl)-1H-benzo[de]isoquinoline-2(3H)-yl)ethyl) carbamate (2c)

The synthesis was conducted similarly to that of the compound 2a using Compound 1e (500 mg, 1.2 mmol), piperazine (1.0 g, 10 eq., 12 mmol) and 50 mL of 2-methoxyethanol to give 420 mg of yellow crystal (yield: 82%).

¹H-NMR (400 MHz, CDCl₃) δ: 8.57 (d, 1H, J=8.5 Hz), 8.51 (d, 1H, J=7.8 Hz), 8.40 (d, 1H, J=7.8 Hz), 7.68 (t, 1H. J=7.8 Hz), 7.20 (d, 1H, J=7.2 Hz), 5.08 (s, 1H), 4.34 (t, 2H, J=7.5 Hz), 3.52-3.51 (m, 2H), 3.23-3.21 (m, 8H), 1.30 (s, 9H); ¹³C-NMR (101 MHz, CDCl₃): δ 164.8, 164.3, 156.5, 156.0, 132.8, 131.3, 130.4, 129.9, 126.1, 125.6, 123.0, 116.3, 114.9, 79.0, 54.3, 46.2, 39.9, 39.6, 28.2.

### Synthesis of tert-butyl (2-(1,3-dioxo-6-(piperazine-1-yl)-1H-benzo[de]isoquinoline-2(3H)-yl)butyl) carbamate (21)

The synthesis was conducted similarly to that of the compound 2a using Compound 1 f (500 mg, 1.1 mmol), piperazine (962 mg, 10 eq., 11 mmol) and 50 mL of 2-methoxyethanol to give 330 mg of yellow crystal (yield: 66%).

¹H-NMR (400 MHz, CDCl₃) δ: 8.57 (d, 1H, J=8.5 Hz), 8.50 (d, 1H, J=7.8 Hz), 8.41 (d, 1H, J=7.8 Hz), 7.69 (t, 1H, J=7.8 Hz), 7.21 (d, 1H, J=7.2 Hz), 4.67 (s, 1H), 4.18 (t, 2H, J=7.5 Hz), 3.25-3.18 (m, 10H), 1.78-1.74 (m, 2H), 1.62-1.58 (m, 2H), 1.42 (s, 9H); ¹³C-NMR (101 MHz, CDCl₃): δ 164.5, 164.0, 156.4, 155.9, 132.6, 131.1, 130.3, 129.9, 126.1, 125.6, 123.2, 116.6, 114.9, 79.0, 54.4, 46.2, 40.2, 39.7, 28.4, 27.5, 25.4.

### Synthesis of tert-butyl (2-(4-(1,3-dioxo-2-propyl-2,3-dihydro-1H-benzo[de]isoquinolin-6-yl))piperazin-1-yl)ethyl) carbamate (3a)

To a 100 mL eggplant-shaped flask, Compound 2a (500 mg, 1.5 mmol), 2-(boc-amino)ethyl bromide (827 mg, 2.5 eq., 3.7 mmol), K₂CO₃ (510 mg, 2.5 eq., 3.7 mmol) and 50 mL of acetonitrile were added and refluxed at 100° C. for 16 hours. After cooling the reaction solution to room temperature, the reaction solution was evaporated using an evaporator. A silica gel column was used for purification using a gradient from 100% chloroform to chloroform/methanol=9/1, 380 mg of yellow crystal was obtained (yield: 54%).

¹H-NMR (400 MHz, CDCl₃) δ: 8.58 (d, 1H, J=7.2 Hz), 8.51 (d, 1H, J=8.5 Hz), 8.39 (d, 1H, J=7.8 Hz), 7.68 (t, 1H, J=7.8 Hz), 7.21 (d, 1H, J=7.8 Hz), 5.01 (s, 1H), 4.13 (t, 2H, J=7.5 Hz), 3.29 (m, 6H), 2.79 (s, 4H), 2.62 (t, 2H, J=7.3 Hz), 1.78-1.73 (m, 2H), 1.47 (s, 9H), 1.00 (t, 3H, J=7.3 Hz); ¹³C-NMR (101 MHz, CDCl₃): δ 164.4, 163.9, 155.9, 155.8, 132.4, 131.0, 130.1, 129.8, 126.1, 125.6, 123.2, 116.7, 114.8, 79.2, 61.9, 57.2, 52.9, 42.3, 41.7, 37.1, 28.4, 23.2, 21.4, 11.5.

### Synthesis of tert-butyl (2-(4-(1,3-dioxo-2-pentyl-2,3-dihydro-1H-benzo[de]isoquinolin-6-yl)piperazin-1-yl)ethyl) carbamate (3b)

The synthesis was conducted similarly to that of Compound 3a using Compound 2b (150 mg, 0.42 mmol), 2-(boc-amino)ethyl bromide (287 mg, 3.0 eq., 1.26 mmol), K₂CO₃ (177 mg, 3.0 eq., 1.26 mmol) and 15 mL of acetonitrile to give 100 mg of yellow crystal (yield: 48%).

¹H-NMR (400 MHz, CDCl₃) δ: 8.58 (d, 1H, J=8.5 Hz), 8.51 (d, 1H, J=7.8 Hz), 8.39 (d, 1H, J=7.8 Hz), 7.68 (t, 1H, J=7.8 Hz), 7.21 (d, 1H, J=7.2 Hz), 4.99 (s, 1H), 4.15 (t, 2H, J=7.5 Hz), 3.29 (s, 6H), 2.78 (s, 4H), 2.62 (m, 2H), 1.72 (m, 2H), 1.47 (s, 9H), 1.39 (s, 2H), 1.39 (s, 4H), 0.91 (t, 3H, J=7.3 Hz); ¹³C-NMR (101 MHz, CDCl₃): δ 164.4, 164.0, 155.9, 155.8, 132.5, 131.0, 130.1, 129.8, 126.1, 125.6, 123.3, 116.8, 114.9, 79.3, 57.2, 53.0, 45.7, 40.3, 37.1, 29.7, 29.2, 28.4, 27.8, 22.4, 14.0.

### Synthesis of tert-butyl (2-(4-(2-heptyl-1,3-dioxo-2,3-dihydro-1H-benzo[de]isoquinoline-6-yl)piperazin-1-yl)ethyl) carbamate (3c)

The synthesis was conducted similarly to that of Compound 3a using Compound 2c (300 mg, 0.79 mmol), 2-(hoc-amino)ethyl bromide (531 mg, 3.0 eq., 2.4 mmol), K₂CO₃ (327 mg, 3.0 eq., 2.4 mmol) and 30 mL of acetonitrile to give 220 mg of yellow crystal (yield: 53%).

¹H-NMR (400 MHz, CDCl₃) δ: 8.56 (d, 1H, J=8.5 Hz), 8.51 (d, 1H, J=7.8 Hz), 8.39 (d, 1H, J=7.8 Hz), 7.68 (t, 1H, J=7.8 Hz), 7.21 (d, 1H, J=7.2 Hz), 4.99 (s, 1H), 4.15 (t, 2H, J 7.5 Hz), 3.29 (s, 6H), 2.78 (s, 4H), 2.62 (t, 2H, J=7.8 Hz), 1.75-1.68 (m, 2H), 1.47 (s, 9H), 1.43-1.28 (m, 8H), 0.87 (t, 3H, J=7.3 Hz); ¹³C-NMR (101 MHz, CDCl₃): δ 164.4, 164.0, 155.9, 155.8, 132.4, 131.0, 130.1, 129.8, 126.1, 125.6, 123.3, 116.8, 114.9, 79.3, 57.2, 53.0, 40.3, 31.7, 29.6, 29.0, 28.4, 28.1, 27.9, 27.1, 23.2, 22.6, 14.0.

### Synthesis of tert-butyl (2-(4-(2-decyl-1,3-dioxo-2,3-dihydro-1H-benzo[de]isoquinoline-6-yl)piperazin-1-yl)ethyl) carbamate (3d)

The synthesis was conducted similarly to that of Compound 3a using Compound 2d (300 mg, 0.71 mmol), 2-(boc-amino)ethyl bromide (478 mg, 3.0 eq., 2.1 mmol), K₂CO₃ (294 mg, 3.0 eq., 2.1 mmol) and 30 mL of acetonitrile to give 200 mg of yellow crystal (yield: 50%).

¹H-NMR (400 MHz, CDCl₃) δ: 8.58 (d, 1H, J=8.5 Hz), 8.51 (d, 1H, J=7.8 Hz), 8.39 (d, 1H, J=7.8 Hz), 7.68 (t, 1H, J=7.8 Hz), 7.21 (d, 1H, J=7.2 Hz), 4.99 (s, 1H), 4.15 (t, 2H, J=7.5 Hz), 3.29 (s, 6H), 2.78 (s, 4H), 2.62 (t, 2H, J=7.8 Hz), 1.75-1.67 (m, 2H), 1.47 (s, 9H), 1.44-1.25 (m, 14H), 0.88 (t, 3H, J=7.3 Hz); ¹³C-NMR (101 MHz, CDCl₃): δ 164.4, 163.9, 155.9, 155.8, 132.4, 131.0 130.1, 129.8, 126.1, 125.6, 123.3, 116.8, 114.8, 79.2, 61.2, 57.2, 53.0, 40.3, 37.1, 31.8, 29.5, 29.4, 29.3, 28.7, 28.4, 28.1, 27.9, 27.1, 22.6, 14.1.

### Synthesis of tert-butyl(2-(6-(4-(2-((tert-butoxycarbonyl)amino) ethyl)piperazin-1-yl)-1,3-dioxo-1H-benzo[de]isoquinoline-2(3H)-yl)ethyl) carbamate (3e)

The synthesis was conducted similarly to that of Compound 3a using Compound 2e (300 mg, 0.71 mmol), 2-(boc-amino)ethyl bromide (475 mg, 3.0 eq., 2.1 mmol), K₂CO₃ (290 mg, 3.0 eq., 2.1 mmol) and 30 mL of acetonitrile to give 220 mg of yellow crystal (yield: 54%).

¹H-NMR (400 MHz, CDCl₃) δ: 8.58 (d, 1H, J=8.5 Hz), 8.52 (d, 1H, J=7.8 Hz), 8.40 (d, 1H, J=7.8 Hz), 7.68 (t, 1H, J=7.8 Hz), 7.21 (d, 1H, J=7.2 Hz), 5.01 (s, 2H), 4.33 (bs, 2H), 3.52-3.51 (m, 2H), 3.29 (s, 6H), 2.79 (s, 4H), 2.62 (bs, 2H), 1.47 (s, 9H), 1.30 (s, 9H); ¹³C-NMR (101 MHz, CDCl₃): δ 164.8, 164.3, 156.0, 155.9, 132.8, 131.3, 130.4, 130.0, 126.1, 125.6, 123.0, 116.5, 114.9, 79.3, 79.0, 57.2, 53.0, 52.9, 39.9, 39.6, 37.1, 28.4, 28.2.

### Synthesis of tert-butyl(2-(6-(4-(2-((tert-butoxycarbonyl)amino) ethyl)piperazin-1-yl)-1,3-dioxo-1H-benzo[de]isoquinoline-2(3H)-yl)butyl) carbamate (3f)

The synthesis was conducted similarly to that of Compound 3a using Compound 2f (150 mg, 0.33 mmol), 2-(boc-amino)ethyl bromide (300 mg, 4.0 eq., 1.32 mmol), K₂CO₃ (69 mg, 1.5 eq., 0.5 mmol) and 15 mL of acetonitrile to give 160 mg of yellow crystal (yield: 81%).

¹H-NMR (400 MHz, CDCl₃) δ: 8.57 (d, 1H, J=8.5 Hz), 8.51 (d, 1H, J=7.8 Hz), 8.40 (d, 1H, J=7.8 Hz), 7.68 (t, 1H, J=7.8 Hz), 721 (d, 1H, J 7.2 Hz), 4.98 (s, 1H), 4.62 (s, 1H), 4.18 (t, 2H, J=7.8 Hz), 3.29 (s, 6H), 3.19-3.18 (m, 2H), 2.79 (s, 4H), 2.62 (t, 2H, J=7.8 Hz), 1.78-1.74 (m, 2H), 1.62-1.58 (m, 2H), 1.47 (s, 9H), 1.42 (s, 9H); ¹³C-NMR (101 MHz, CDCl₃): δ 164.5, 164.0, 155.9, 132.6, 131.1, 130.3, 129.8, 126.1, 125.6, 123.2, 116.7, 114.9, 79.3, 79.0, 572, 53.0, 40.2, 39.7, 37.1, 28.4, 27.5, 25.4.

### Synthesis of 2-(4-(1,3-dioxo-2-propyl-2,3-dihydro-1H-benzo[de]isoquinolin-6-yl) piperazin-1-yl) ethane-1-amine hydrochloride salt (4a)

To a 50 mL eggplant-shaped flask, Compound 3a (150 mg, 0.32 mmol) and 5 mL of THF were added and dissolved. To the mixture, 5 mL of 4 N HCl/dioxane was added and stirred at room temperature for 2 hours. The crystal precipitated was filtrated and washed with THF and CHCl₃ to give 100 mg of yellow crystal (yield: 77%).

¹H-NMR (400 MHz, CD₃OD) δ: 8.61-8.54 (m, 3H), 7.86 (t, 1H, J=8.3 Hz), 7.50 (d, 1H, J=7.3 Hz), 4.12 (t, 2H, J=7.3 Hz), 3.71-3.56 (m, 12H), 1.80-1.71 (m, 2H), 1.01 (t, 3H, J=7.3 Hz); ¹³C-NMR (101 MHz, CD₃OD): δ 164.2, 163.7, 153.7, 131.9, 130.9, 129.8, 129.3, 126.3, 126.0, 122.9, 117.8, 115.8, 53.2, 52.5, 49.6, 41.3, 33.7, 20.9, 10.3.

### Synthesis of 2-(4-(1,3-dioxo-2-pentyl-2,3-dihydro-1H-benzo[de]isoquinolin-6-yl)piperazin-1-yl)ethane-1-amine hydrochloride salt (4b)

Synthesis was conducted similarly to that of Compound 4a using Compound 3b (130 mg, 0.26 mmol), 5 mL of THF and 5 mL of 4N HCl/dioxane to give 90 mg of yellow crystal (yield: 80%).

¹H-NMR (400 MHz, CD₃OD) δ: 8.62-8.54 (m, 3H), 7.87 (t, 1H, J=8.3 Hz), 7.50 (d, 1H, J=7.3 Hz), 4.15 (t, 2H, J=7.3 Hz), 3.68-3.50 (m, 12H), 1.73 (t, 2H, J=8.3 Hz), 1.42 (s, 4H), 0.95 (t, 3H, J=7.3 Hz); ¹³C-NMR (101 MHz, CD₃OD): δ 164.1, 163.7, 153.7, 131.9, 130.9, 129.8, 129.3, 126.3, 126.0, 122.9, 117.8, 115.8, 53.2, 52.5, 49.6, 39.8, 33.7, 28.9, 27.3, 22.0, 12.9.

### Synthesis of 2-(4-(2-heptyl-1,3-dioxo-2,3-dihydro-1H-benzo[de]isoquinolin-6-yl)piperazin-1-yl)ethane-1-amine hydrochloride salt (4c)

Synthesis was conducted similarly to that of Compound 4a using Compound 3c (130 mg, 0.24 mmol), 5 mL of THF and 5 mL of 4N HCl/dioxane to give 80 mg of yellow crystal (yield: 70%).

¹H-NMR (400 MHz, CD₃OD) δ: 8.50 (d, 1H, J=8.3 Hz), 8.43 (d, 1H, J=8.3 Hz), 7.80 (t, 1H, J=8.3 Hz), 7.42 (d, 1H, J=7.3 Hz), 4.08 (t, 2H, J=7.3 Hz), 3.87-3.59 (m, 12H), 1.68 (t, 2H, J=8.3 Hz), 1.41-1.32 (m, 8H), 0.91 (t, 3H, J=7.3 Hz); ¹³C-NMR (101 MHz, CD₃OD): δ 164.1, 163.7, 153.7, 131.8, 130.8, 129.8, 129.3, 126.3, 126.0, 122.8, 117.8, 115.7, 53.2, 52.5, 49.6, 39.8, 33.7, 31.5, 28.7, 27.6, 26.7, 22.2, 13.0.

### Synthesis of 2-(4-(2-decyl-1,3-dioxo-2,3-dihydro-1H-benzo[de]isoquinolin-6-yl)ethane-1-amine hydrochloride salt (4d)

Synthesis was conducted similarly to that of Compound 4a using Compound 3d (130 mg, 0.23 mmol), 5 mL of THF and 5 mL of 4N HCl/dioxane to give 80 mg of yellow crystal (yield: 69%).

¹H-NMR (400 MHz, CD₃OD) δ: 8.56-8.53 (m, 2H), 8.49 (d, 1H, J=8.3 Hz), 7.84 (t, 1H, J=8.3 Hz), 7.46 (d, 1H, J=7.3 Hz), 4.20 (t, 2H, J=7.3 Hz), 3.90-3.58 (m, 12H), 1.74-1.67 (m, 2H), 1.41-1.30 (m, 14H), 0.90 (t, 3H, J=7.3 Hz); ¹³C-NMR (101 MHz, CD₃OD): δ 164.1, 163.7, 153.7, 137.7, 131.8, 130.8, 129.8, 129.3, 128.1, 126.3, 126.0, 124.7, 122.9, 117.8, 115.7, 53.3, 52.5, 49.6, 39.8, 33.9, 33.7, 31.6, 29.4, 29.2, 29.0, 27.6, 27.2, 26.7, 22.3, 13.0.

### Synthesis of 2-(4-(2-(2-((tert-butoxycarbonyl)amino)ethyl)-1,3-dioxo-2,3-dihydro-1H-benzo[de]isoquinolin-6-yl)piperazin-1-yl)ethane-1-amine hydrochloride salt (4e)

Synthesis was conducted similarly to that of Compound 4a using Compound 3e (120 mg, 0.21 mmol), 5 mL of THF and 5 mL of 4N HCl/dioxane to give 70 mg of yellow crystal (yield: 75%).

¹H-NMR (400 MHz, CD₃OD) δ: 8.67-8.59 (m, 3H), 7.90 (t, 1H, J=8.3 Hz), 7.52 (d, 1H, J=7.3 Hz), 4.49 (t, 2H, J=7.3 Hz), 3.71-3.57 (m, 12H).

### Synthesis of 2-(4-(2-(2-((tert-butoxycarbonyl)amino)ethyl)-1,3-dioxo-2,3-dihydro-1H-benzo[de]isoquinolin-6-yl)piperazin-1-yl)butane-1-amine hydrochloride salt (4f)

Synthesis was conducted similarly to that of Compound 4a using Compound 3f (40 mg, 0.067 mmol), 5 mL of THF and 5 mL of 4N HCL/dioxane to give 20 mg of yellow crystal (yield: 64%).

¹H-NMR (400 MHz, CD₃OD) δ: 8.63-8.56 (m, 3H), 7.88 (t, 1H, J=8.3 Hz), 7.51 (d, 1H, J=7.3 Hz), 4.22 (t, 2H, J=7.3 Hz), 3.74-3.57 (m, 12H), 3.03 (t, 2H, J=7.3 Hz), 1.87-1.75 (m, 4H); ¹³C-NMR (101 MHz, DMSO-d₆): δ 164.0, 163.5, 154.3, 132.5, 131.3, 130.9, 129.4, 127.0, 125.8, 123.1, 117.2, 116.3, 53.4, 51.9, 49.7, 33.8, 25.2, 25.1.

### Synthesis of tert-butyl (5-((1,3-dioxo-2-pentyl-2,3-dihydro-1H-benzo[de]isoquinoline-6-yl)amino)pentyl) carbamate (5h)

To a 200 mL eggplant-shaped flask, Compound 1b (300 mg, 0.86 mmol), tert-butyl (5-aminopentyl) carbamate (210 mg, 1.2 eq., 1.0 mmol) and 40 mL of 2-methoxyethanol were added and refluxed at 120° C. for 16 hours. The progress of the reaction was checked and the reaction solution was evaporated using an evaporator. A silica gel column was used for purification using a gradient from 100% chloroform to chloroform/methanol=9/1, 310 mL of pale yellow oil was obtained (yield: 77%).

¹H-NMR (400 MHz, CDCl₃) δ: 8.58 (d, 1H, J=7.2 Hz), 8.46 (d, 1H, J=8.5 Hz), 8.17 (d, 1H, J=7.8 Hz), 7.61 (t, 1H, J=7.8 Hz), 6.70 (d, 1H, J=7.8 Hz), 5.38 (bs, 1H), 4.54 (bs, 1H), 4.15 (t, 2H, J=7.5 Hz), 3.43-3.39 (m,), 3.22-3.10 (m), 1.85 (t), 1.72 (t), 1.59-1.25 (m), 0.90 (t, 3H).

### Synthesis of tert-butyl (2-((2-((1,3-dioxo-2-pentyl-2,3-dihydro-1H-benzo[de]isoquinolin-6-yl)amino)ethyl)(methyl)amino)ethyl)carbamate (5i)

Similarly to that of Compound 5h, synthesis was conducted using Compound 1b (200 mg, 0.57 mmol), tert-butyl (2-((2-aminoethyl)(methyl)amino)ethyl) carbamate (188 mg, 1.5 eq., 0.86 mmol) and 20 mL of 2-methoxyethanol to give 120 mg of pale yellow oil (yield: 43%).

### Synthesis of 2-(4-(1,3-dioxo-2-propyl-2,3-dihydro-1H-benzo[de]isoquinolin-6-yl)piperazin-1-yl)ethane-1-amine hydrochloride salt (6h)

To a 50 mL eggplant-shaped flask, Compound 5h (100 mg, 0.86 mmol) and 10 mL of THF were added and dissolved. To the mixture, 10 mL of 4 N HCl/dioxane was added and stirred at room temperature for 2 hours. The crystal precipitated was filtrated and washed with THF and CHCl₃ to give 20 mg of yellow crystal (yield: 23%).

¹H-NMR (400 MHz, CD₃OD) δ: 8.57-8.52 (m, 2H), 8.38 (d, 1H, J=7.3 Hz), 7.67 (t, 1H, J=8.3 Hz), 6.82 (d, 1H, J=7.3 Hz), 4.12 (t, 2H, J=7.3 Hz), 3.51 (t, 2H, J=7.3 Hz), 2.97 (t, 2H), 1.89-1.85 (m, 2H), 1.79-1.69 (m, 2H), 1.63-1.57 (m, 2H), 1.54-1.46 (m, 2H), 1.41 (m, 4H), 0.95 (t, 3H, J=7.3 Hz).

### Synthesis of 2-(4-(1,3-dioxo-2-propyl-2,3-dihydro-1H-benzo[de]isoquinolin-6-yl) piperazin-1-yl) ethane-1-amine hydrochloride salt (6i)

To a 50 mL eggplant-shaped flask, Compound 5i (100 mg, 0.2 mmol) and 10 mL of THF were added and dissolved. To the mixture, 10 mL of 4 N HCl/dioxane was added and stirred at room temperature for 2 hours. The crystal precipitated was filtrated and washed with THF and CHCl₃ to give 20 mg of yellow crystal (yield: 23%).

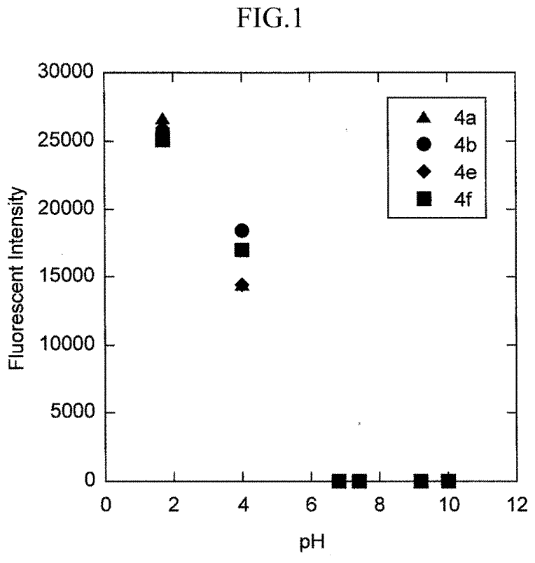

As shown in FIG. 1, it was confirmed that the fluorescent intensities of Compounds 4a, 4b, 4c and 4f increase under acidic condition of pH less than 6.

### Example 2: Autophagy Detection Test (1)

(1) Introduction of Fluorescent Compound to Cell and Induction or Inhibition of Autophagy

HeLa cells were seeded on a μ-slide 8 well (Ibidi) and incubated in a CO₂ incubator at 37° C. overnight. Compound 4b (1 μM) or Compound 6h (0.1 μM) diluted with serum medium was added and incubated for 30 minutes. After washing twice with serum medium, the cells were incubated at 37° C. for 6 hours or 20 hours in an amino acid-free medium of serum containing-medium and observed with a fluorescent microscope. As an autophagy inducing agent, 0.5 μM Rapamycin was used and as an autophagy inhibiting agent, 10 μM chloroquine and 0.1 μM bafilomycin A1 were used.

(2) Evaluation using Flow Cytometry

HeLa cells were seeded on a 24 well plate and incubated in a CO₂ incubator at 37° C. overnight, 1 μM of Compound 4b diluted with serum medium was added and incubated for 30 minutes. After washing twice with the serum-free medium, a medium free from amino acid and serum was added and the cells were incubated at 37° C. for 6 hours or 20 hours. Cell were washed with PBS once, peeled with 0.25% trypsin-EDTA and used for flow cytometric analysis.

(3) Western Blot Analysis

Cells induced under arbitrary conditions were washed once with PBS and recovered using Lysis buffer containing protease inhibitor. The cells were separated using 15% polyacrylamide gel and transferred onto a PVDF membrane. After blocking, the membrane was immersed in Anti-LC3 antibody (MBL) diluted to 1,000 folds and incubated at 4° C. for 16 hours. The PVDF membrane was washed with PBST, immersed in HRP binding antibody diluted to 10,000 folds and shaken for 1 hour at room temperature. After washing 3 times with PBST, the cells were detected using chemiluminescence.

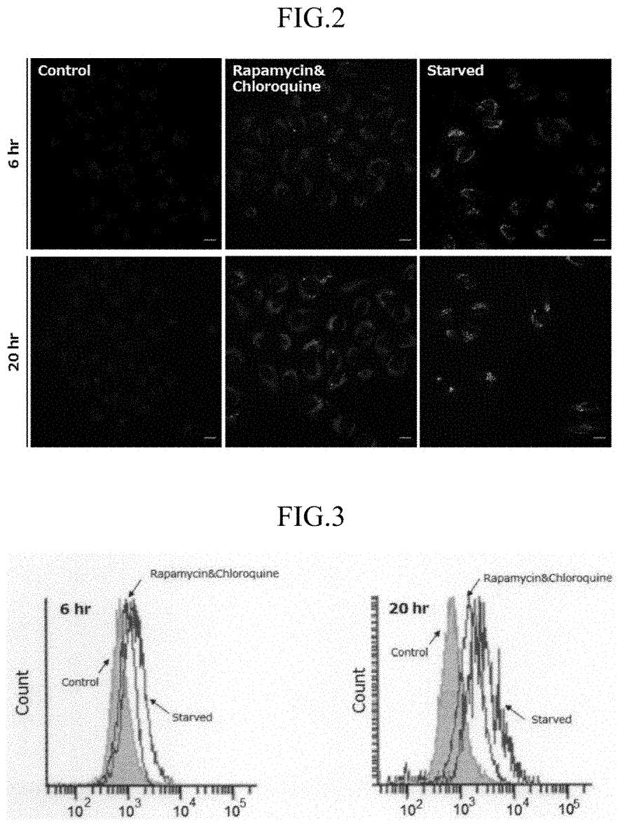

Fluorescent microscopic images of HeLa cells after introducing the Compound 4b and incubating for 6 hours and 20 hours are shown in FIG. 2. In FIG. 2, “Control” shows a result of control group. In both of the group in which autophagy was induced by adding Rapamycin (“Rapamycin & Chloroquine”) and the group in which autophagy was induced starvation culture (“Starved”), enhancement of the fluorescent intensities was observed.

Results of flow cytometric measurement of HeLa cells after introducing the Compound 4b and incubating for 6 hours and 20 hours under starvation condition are shown in FIG. 3. It was confirmed that Compound 4b is useful for detecting autophagy using not only fluorescent microscopy but also flow cytometry.

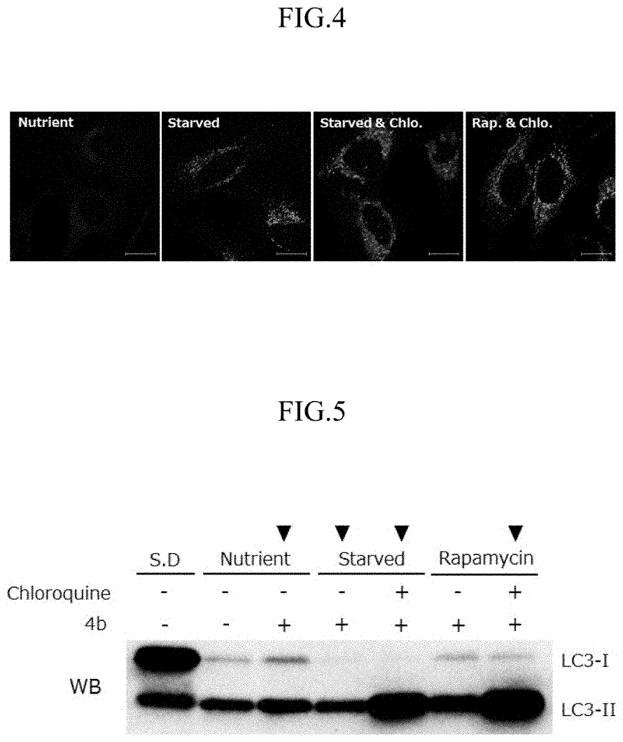

Changes of the fluorescent intensities in HeLa cells of which autophagy was induced by Rapamycin in the presence of chloroquine (“Rapamycin & Chloroquine”) or starvation induction (“Starved”) are shown in FIG. 4. In FIG. 4, “Nutrient” shows a measurement result of HeLa cells incubated in a normal nutrient medium as a control group. Similarly to the result shown in FIG. 2, enhancement of the fluorescent intensities in accordance with the induction of autophagy are confirmed and it is recognized that the enhancement of the fluorescent intensity is correlated with the result of quantitative analysis of the expression amount of LC3, an autophagy marker using Western blotting shown in FIG. 5

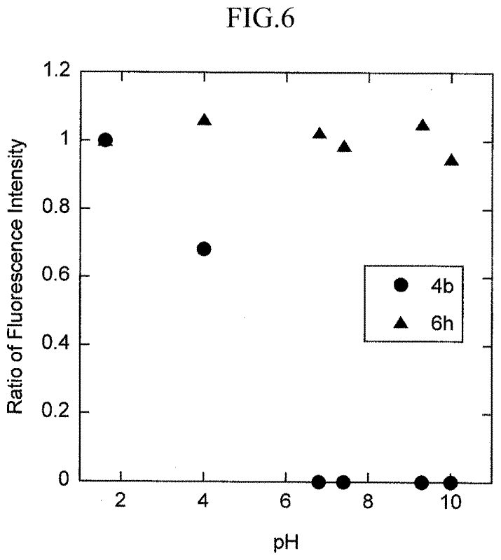

FIG. 6 shows a graph showing pH dependence of fluorescent intensities of Compounds 4b and 6h. It was confirmed that the fluorescent intensity of Compound 6h shows no pH dependence, which is different from Compound 4b. These results show that in Compound 6h having no nitrogen atom in the side chain, the fluorescent intensity does not decrease under the condition of pH 6 or more since no photo induced electron transfer takes place.

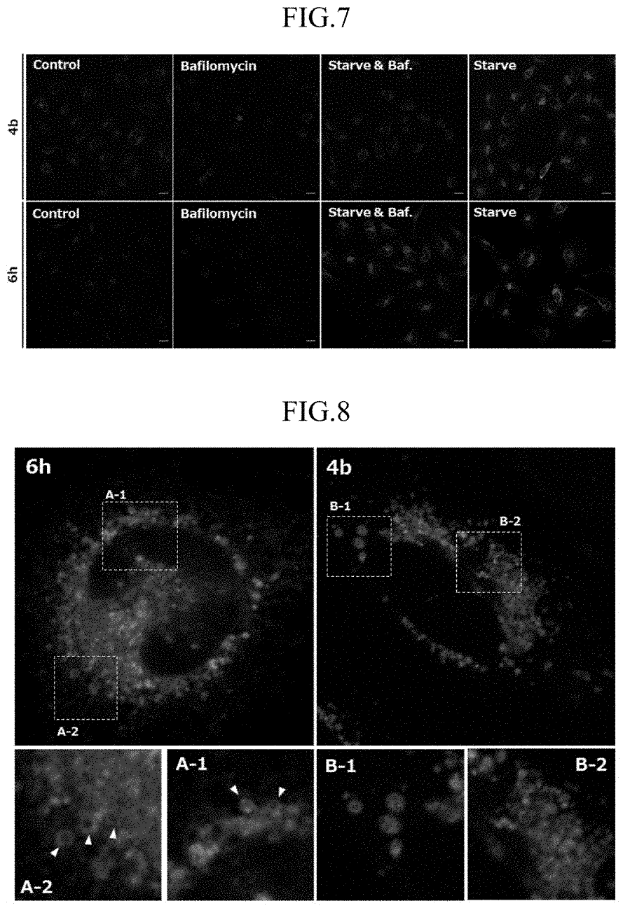

FIG. 7 shows fluorescent microscopic images showing the result of the comparison of the fluorescent intensities of Compound 4b and Compound 6h introduced in HeLa cells. In the control group (“Control”) and in the presence of Bafilomycin, an ATPase inhibitor which inhibits the fusion of autophagosomes and lysosomes (“Bafilomycin”) in which autophagy hardly occurs, the fluorescent intensities of both compounds were low, whereas under the starvation condition (“Starved”) in which autophagy was induced, both compounds showed high fluorescent intensities. On the other hand, when bafilomycin was added under starvation condition, the fluorescent intensity of Compound 4b decreased whereas the fluorescent intensity of Compound 6h did not decrease. These results show that Compound 6h shows fluorescent emission even in the autophagosomes not fused with the lysosomes independent from pH.

FIG. 8 shows a high-magnification fluorescent microscopic images Compound 4b and Compound 6h introduced in HeLa cells under the condition in which autophagy was induced. Evidently from partial expanded views of the fluorescent microscopic image indicated in A-1 and A-2, it was confirmed that Compound 6h shows ring-shaped fluorescent emission. This result shows that Compound 6h only stains autophagosome membrane. From these results, Compound 6h is expected to show the response from the step of the formation of the autophagosome.

By using the Fluorescent Compound 4b of which fluorescent intensity shows pH dependence and the Fluorescent Compound 6h of which fluorescent intensity does not show pH dependence, there is a possibility of the detection of the formation of the autophagosome (early stage of autophagy) and the autolysosome in the decomposition step (late stage of autophagy) stepwise. By combining the method known in the art, investigation of the mechanism of autophagy and the relation with organelle in the cells may also be expected.

### Example 3: Preparation of Fluorescent Compound (2)

### Synthesis of 2-pentyl-1H-benzo[10,5]anthra[2,1,9-def]isoquinolin-1,3(2H)-dione (7)

To a 200 mL eggplant-shaped flask, perylene-3,4-dicaboxylic anhydride (1 g, 3.1 mmol), propyl amine (322 mg, 1.2 eq., 3.7 mmol), DMAP (452 mg, 1.2 eq., 3.7 mmol) and 100 mL of DMF were added and refluxed at 90° C. for 16 hours. After cooling to room temperature, crystal precipitated was filtrated to give 1 g of red crystal (yield: 82%).

¹H-NMR (400 MHz, CDCl₃) δ: 8.40 (d, 2H), 8.24 (d, 2H), 8.18 (d, 2H), 7.81 (d, 2H), 7.55 (d, 2H), 4.16 (t, 2H), 1.78-1.75 (m, 2H), 1.46-1.42 (m, 4H), 0.94 (t, 3H).

### Synthesis of 8-bromo-2-hexyl-1H-benzo[10,5]anthra[2,1,9-def]isoquinolin-1,3(2H)-dione (8)

To a 200 mL eggplant-shaped flask, Compound 7 (1g, 2.5 mmol) and 100 mL of 1,2-dichloroethane were added and dissolved. Bromine was added and the mixture was refluxed for 16 hours to give 800 mg of black crystal (yield: 68%).

¹H-NMR (400 MHz, CDCl₃) δ: 8.56 (t, 2H), 8.44 (d, 1H), 8.38 (d, 1H), 8.33 (d, 1H), 8.29 (d, 1H), 8.18 (d, 1H), 7.89 (d, 1H), 7.70 (t, 1H), 4.19 (t, 2H), 1.76 (t, 2H), 1.42 (m, 4H), 0.93 (t, 3H).

### Synthesis of 2-pentyl-8-(piperazin-1-yl)-1H-benzo[10,5]anthra[2,1,9-def]isoquinolin-1,3(2H)-dione (9)

In a 200 mL eggplant-shaped flask, synthesis was conducted using Compound 8 (300 mg, 0.63 mmol), piperazine (542 mg, 10 eq., 6.3 mmol) and 100 mL of 2-methoxyethanol to give 300 mg of black crystal (yield: 95%).

¹H-NMR (400 MHz, CDCl₃) δ: 8.52 (t, 2H), 8.41 (d, 1H), 8.34 (t, 2H), 8.24 (t, 2H), 7.62 (t, 1H), 7.19 (d, 1H), 4.19 (t, 2H), 3.22 (m, 8H), 1.70 (m, 2H), 1.42 (m, 4H), 0.92 (t, 3H).

### Synthesis of tert-butyl (2-(4-(1,3-dioxo-2-pentyl-2,3-dihydro-1H-benzo[10,5]anthra[2,1,9-def]isoquinoline-8-yl)piperazin-1-yl)ethyl) carbamate (10)

In a 100 mL eggplant-shaped flask, the synthesis was conducted using Compound 9 (300 mg, 0.63 mmol), 2-(boc-amino)ethyl bromide (212 mg, 1.5 eq., 0.94 mmol), K₂CO₃ (130 mg, 1.5 eq., 0.94 mmol) and 50 mL of acetonitrile to give 100 mg of black crystal (yield: 25%).

¹H-NMR (400 MHz, CDCl₃) δ: 8.54 (t, 2H), 8.43 (d, 1H), 8.37-8.34 (dd, 2H), 8.28 (d, 1H), 8.22 (d, 1H), 7.62 (t, 1H), 7.20 (d, 1H), 5.03 (s, 1H), 4.19 (t, 2H), 3.34-3.25 (m, 6H), 2.80 (s, 4H), 2.63 (t, 2H), 1.76 (m, 2H), 1.60 (s, 8H), 1.49 (s, 9H), 1.42 (m, 5H), 1.33-1.25 (m, 6H), 0.91 (t, 3H); ¹³C-NMR (101 MHz, CDCl₃): δ 164.0, 156.0, 152.6, 137.4, 137.3, 131.5, 131.3, 129.8, 129.5, 129.0, 128.8, 126.7, 126.2, 124.6, 124.0, 123.9, 120.6, 119.7, 118.9, 115.7, 57.3, 53.2, 52.9, 40.4, 37.1, 29.6, 29.5, 29.3, 29.1, 28.4, 27.8, 24.8, 22.6, 22.5, 14.1, 14.0.

### Synthesis of 8-(4-(2-aminoethyl)piperazin-1-yl)-2-pentyl-1H-benzo[10,5]anthra[2,1,9-def]isoquinolin-1,3(2H)-dione (11)

In a 50 mL eggplant-shaped flask, the synthesis was conducted using Compound 10 (100 mg, 0.16 mmol), 5 mL of THF and 5 mL 4N HCL/dioxane to give 30 mg of black crystal (yield: 33%).

¹H-NMR (400 MHz, CD₃OD) δ: 8.34 (d, 1H), 8.27 (d, 1H), 8.23-8.13 (m, 4H), 7.61 (t, 1H), 7.30 (d, 1H), 4.10 (1, 2H), 3.74-3.49 (m, 12H), 1.74 (t, 2H), 1.46-1.45 (m, 4H), 0.99 (t, 3H).

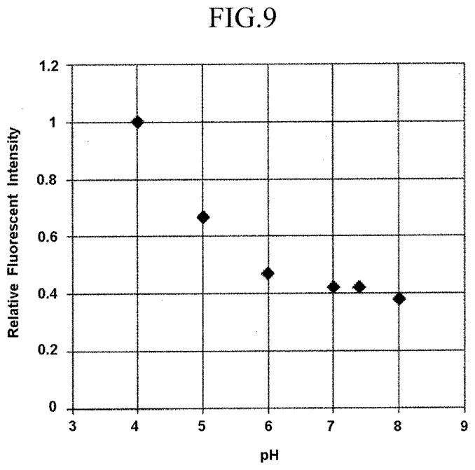

As shown in FIG. 9, it was confirmed that the fluorescent intensity of Compound 11 increases under acidic condition of pH 6 or less.

### Synthesis of tert-butyl (5-((1,3-dioxo-2-pentyl-2,3-dihydro-1H-benzo[10,5]anthra[2,1,9-dej]isoquinoline-8-yl) amino)pentyl) carbamate (12)

In a 200 mL eggplant-shaped flask, the synthesis was conducted using Compound 8 (300 mg, 0.63 mmol), tert-butyl (2-((2-aminoethyl)(methyl)amino)ethyl) carbamate (210 mg, 1.5 eq., 0.94 mmol) and 30 mL of 2-methoxyethanol to give 100 mg of deep purple oil (yield: 19%).

¹H-NMR (400 MHz, CDCl₃) δ: 8.21-8.20 (m, 3H), 7.92 (d, 1H), 7.85 (d, 2H), 7.62 (d, 1H), 7.47 (t, 1H), 6.63 (d, 1H), 3.30-3.14 (m, 6H), 1.67-1.50 (m, 6H), 1.42 (s, 9H), 1.29-1.28 (m, 4H), 0.88 (t, 3H).

### Synthesis of 5-((1,3-dioxo-2-pentyl-2,3-dihydro-1H-benzo[10,5]anthra[2,1,9-def]isoquinolin-8-yl)amino)pentane-1-amine hydrochloric salt (13)

In a 50 mL eggplant-shaped flask, the synthesis was conducted using Compound 12 (100 mg, 0.16 mmol), 5 mL of THF and 5 mL 4N HCL/dioxane to give 30 mg of black crystal (yield: 33%).

¹H-NMR (400 MHz, CD₃OD) δ: 8.23-8.22 (m, 3H), 7.94 (d, 1H), 7.86 (d, 2H), 7.65 (d, 1H), 7.46 (t, 1H), 6.66 (d, 1H), 3.35-3.17 (m, 6H), 1.68-1.49 (m, 6H), 1.33-1.29 (m, 4H), 0.92 (t, 3H).

It was confirmed that the fluorescent intensity of Compound 13 shows no pH dependence similarly to that of Compound 6h.

### Example 4: Autophagy Detection Test (2)

HeLa cells were seeded on a μ-slide 8 well (Ibidi) and incubated in a CO₂ incubator at 37° C. overnight. Compound 6h diluted with a serum medium (0.1 μM) was added and incubated for 30 minutes. After washing twice with serum medium, the cells were incubated at 37° C. for 5 hours in an amino acid-free medium of serum containing-medium and observed with a fluorescent microscope.

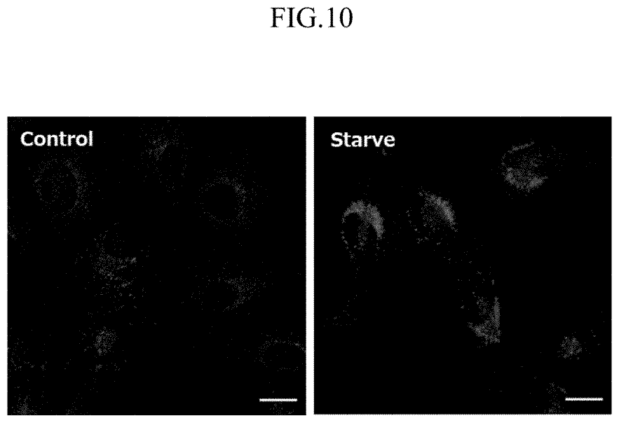

Fluorescent microscopic images of HeLa cells after introducing the Compound 6h and incubating for 5 hours are shown in FIG. 10. In FIG. 10, “Control” shows a result of control group. In the cells incubated under starvation condition (“Starve”) to induce autophagy, enhancement of the fluorescent intensity was observed.

### Example 5: Autophagy Detection Test (3)

HeLa cells were seeded on a μ-slide 8 well (Ibidi) and incubated in a CO₂ incubator at 37° C. overnight. Compound 6h (1 μM) and Compound 11 (0.1 μM) diluted with serum medium was added and incubated for 30 minutes. After washing twice with serum medium, the cells were incubated at 37° C. for 3 hours or 6 hours in an amino acid-free medium of serum containing-medium and observed with a fluorescent microscope.

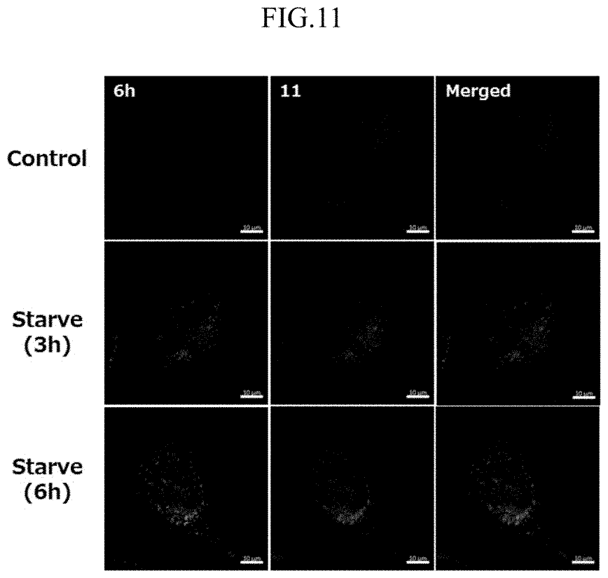

Fluorescent microscopic images of HeLa cells after introducing the Compound 6h and Compound 11 and incubating for 3 hours and 6 hours are shown in FIG. 11. In FIG. 11, “Control” shows a result of control group. In the cells incubated under starvation condition (“Starved”) to induce autophagy, Compound 6h and Compound 11 were introduced in similar cells and incubated under starvation condition. As a result, granular points stained only by Compound 6h and granular points co-stained by Compound 6h and Compound 11 are observed. These results show that autophagosome forming step and autolysosome forming step may be distinguished stepwise by using two types of the fluorescent compounds having different fluorescent wavelength and pH responsivity.

### Example 6: Autophagy Detection Test (4)

HeLa cells were seeded on a μ-slide 8 well (Ibidi) and incubated in a CO₂ incubator at 37° C. overnight. Compound 4b (1 μM) or Compound 13 (0.1 μM) diluted with serum medium was added and incubated for 30 minutes. After washing twice with serum medium, the cells were incubated at 37° C. for 3 hours or 6 hours in an amino acid-free medium of serum containing-medium and observed with a fluorescent microscope.

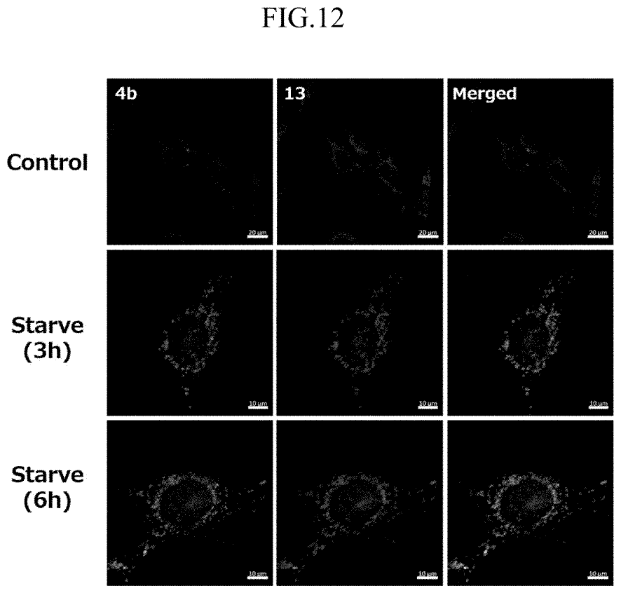

Fluorescent microscopic images of HeLa cells after introducing the Compound 4b and Compound 13 and incubating for 3 hours and 6 hours are shown in FIG. 12. In FIG. 12, “Control” shows a result of control group. In the cells incubated under starvation condition (“Starved”) to induce autophagy, Compound 4b and Compound 13 were introduced in similar cells and incubated under starvation condition. As a result, granular points stained only by Compound 4b and granular points co-stained by Compound 4b and Compound 13 are observed. These results show that autophagosome forming step and autolysosome forming step may be distinguished stepwise by using two types of the fluorescent compounds having different fluorescent wavelength and pH responsivity.

Various embodiments and variations of the invention may be possible without departing from the broad spirit and scope of the invention. The embodiments and examples as mentioned above are provided for illustrating the invention, not for limiting the scope of the invention. In other words, the scope of the invention is defined by attached Claims, not by the embodiments and examples. In addition, various variations made within the scope of the Claims and within the scope of the equivalent of the invention should be within the scope of the invention.

The present application claims the priority based on Japanese Patent Application 2017-100197 filed on May 19, 2017 including the specification, claims, drawings and abstract thereof. The entire disclosure in the Japanese Patent Application mentioned above is to be incorporated into the disclosure by reference.

1: A fluorescent compound or a salt thereof represented by General Formula (I) shown below: 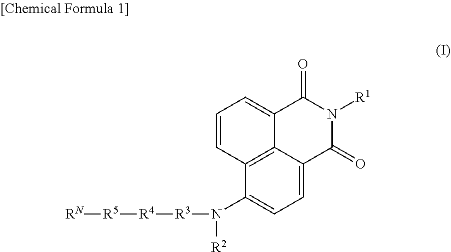
in the General Formula (I) shown above,
R¹ represents an alkyl group or ω-aminoalkyl group,
R² represents a hydrogen atom or an alkyl group,
R³ represents an atomic group represented by a formula —(CH₂)ₘ— (m is a natural number of 10 or less),
R⁴ represents an atomic group represented by a formula —CH₂— or —NR⁶— (R⁶ represents an alkyl group),
R⁵ represents an atomic group represented by a formula —(CH₂)ₙ— (n is a natural number of 10 or less),
R^{N} is an atomic group represented by any one of formulae —NH₂, —NHR⁷, —NR⁷R⁸ and —N⁺R⁷R⁸R⁹ (R⁷, R⁸ and R⁹ independently represent an alkyl group, respectively),
when R² is the alkyl group and R⁴ is an atomic group represented by the formula —NR⁶—, R² and R⁶ may bind with each other to form a ring.

2: The fluorescent compound or the salt thereof according to claim 1, wherein the compound is represented by any one of Formulae 4a to 4f, 6h and 6i shown below.

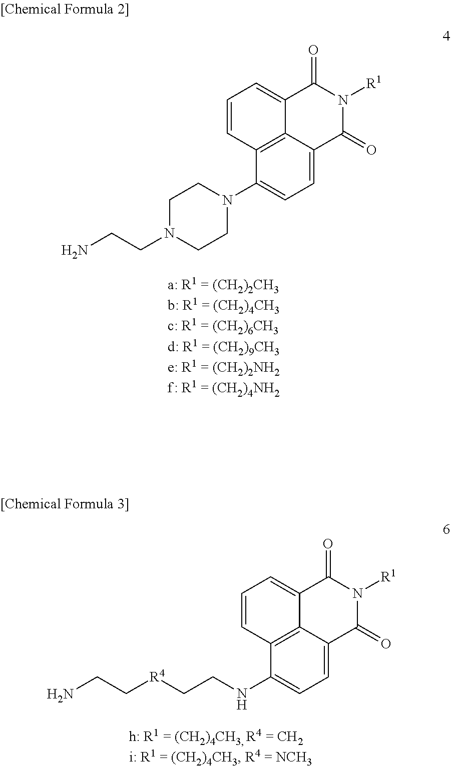

3: The fluorescent compound or the salt thereof according to claim 2, wherein the compound is represented by Formula 4b or 6h shown below.

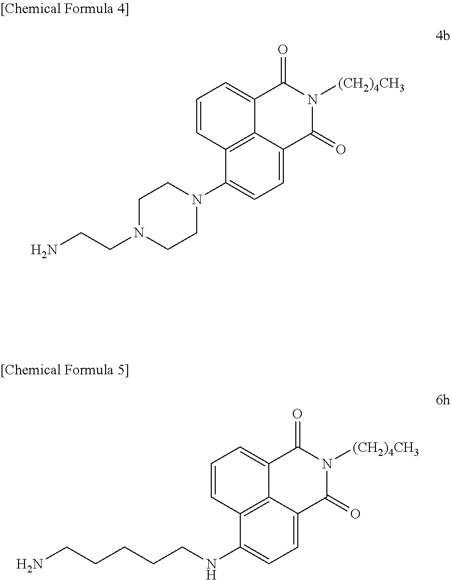

4: A fluorescent compound or a salt thereof represented by General Formula (II) shown below: 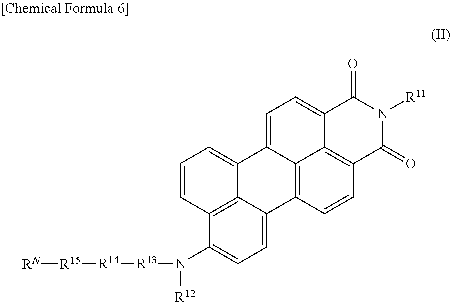
in the General Formula (II) shown above,
R¹¹ represents an alkyl group or an ω-aminoalkyl group,
R¹² represents a hydrogen atom or an alkyl group,
R¹³ represents an atomic group represented by a formula —(CH₂)ₘ— (m is a natural number of 10 or less),
R¹⁴ represents an atomic group represented by a formula —CH₂— or —NR¹⁶— (R¹⁶ represents an alkyl group),
R¹⁵ represents an atomic group represented by a formula —(CH₂)ₙ— (n is a natural number of 10 or less),
R^{N} is an atomic group represented by any one of formulae —NH₂, —NHR¹⁷, —NR¹⁷R¹⁸ and —N⁺R¹⁷R¹⁸R¹⁹ (R¹⁷, R¹⁸, R¹⁹, respectively independently represents alkyl groups),
when R¹² is the alkyl group and R¹⁴ is the atomic group represented by the formula —NR¹⁶—, R¹² and R¹⁶ may bind with each other to form a ring.

5: The fluorescent compound or the salt thereof according to claim 4, wherein the compound is represented by Formula 11 or 13 shown below.

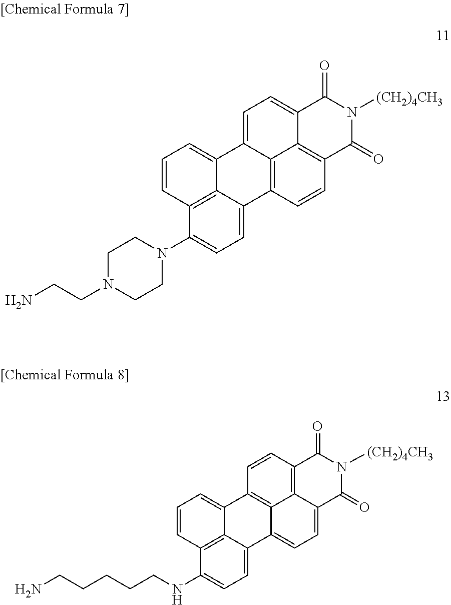

6: An autophagy detection reagent comprising one or more selected from the group consisting of the fluorescent compounds and the salts thereof according to claim 1.

7: The autophagy detection reagent according to claim 6, wherein the reagent comprises one or more selected from a group consisting of fluorescent compounds and salts thereof represented by the Formulae 4a to 4f shown below and one or more selected from a group consisting of fluorescent compound represented by the Formulae 6h and 6i shown below and salts thereof.

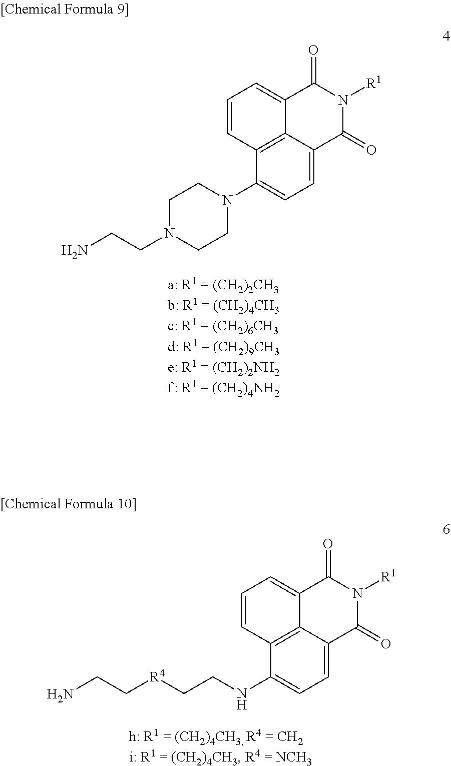

8: The autophagy detection reagent according to claim 7, wherein the reagent comprises a fluorescent compound represented by the Formula 4b shown below or a salt thereof and a fluorescent compound represented by the Formula 6h or a salt thereof.

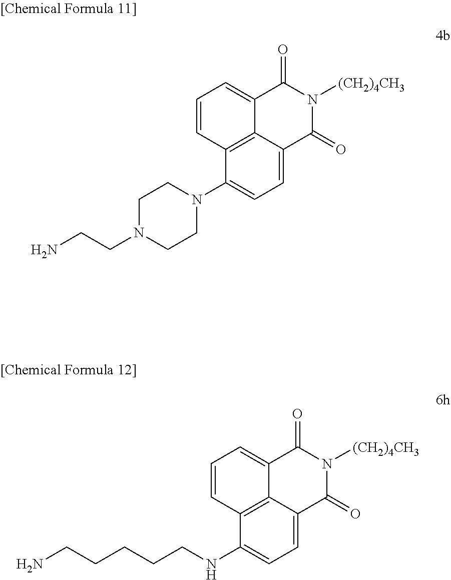

9: The autophagy detection reagent according to claim 6 comprising a fluorescent compound represented by Formula 11 shown below or a salt thereof and a fluorescent compound represented by Formula 13 shown below or a salt thereof.

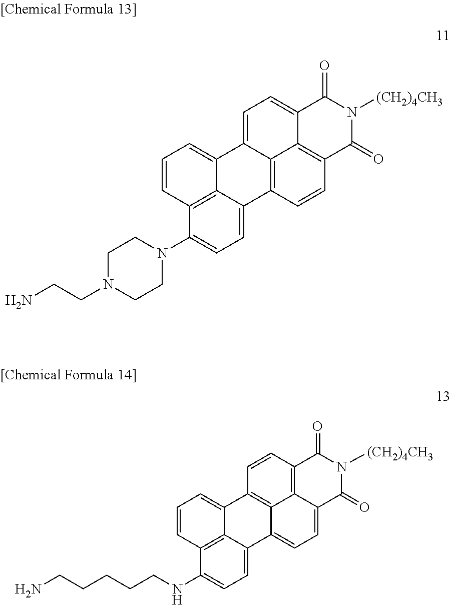

10: The autophagy detection reagent according to claim 6, wherein the reagent comprises one or more selected from a group consisting of fluorescent compounds and salts thereof represented by General Formula (Ia) shown below and a salt thereof and one or more selected from a group consisting of fluorescent compounds represented by General Formula (IIb) and a salt thereof; or
one or more selected from a group consisting of fluorescent compounds and salts represented by General Formula (Ib) shown below and salt thereof and one or more selected from a group consisting of fluorescent compounds represented by General Formula (IIa) and a salt thereof. 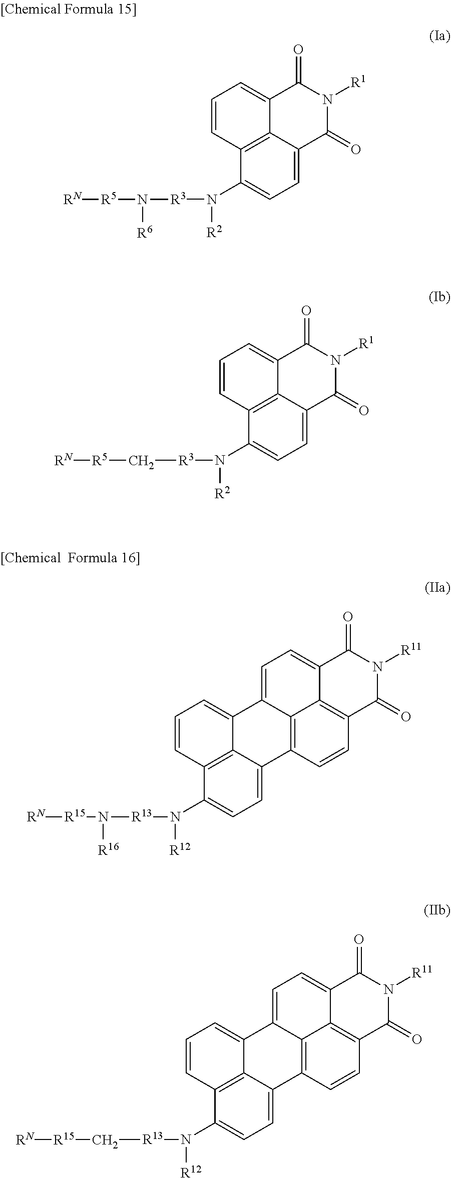
in the General Formulae (Ia) and (Ib) shown above,
R¹ represents an alkyl group or ω-aminoalkyl group,
R² represents a hydrogen atom or an alkyl group,
R³ represents an atomic group represented by a formula —(CH₂)ₘ— (m is a natural number of 10 or less),
R⁵ represents an atomic group represented by a formula —(CH₂)ₙ— (n is a natural number of 10 or less),
R^{N} is an atomic group represented by any one of formulae —NH₂, —NHR⁷, —NR⁷R⁸ and —N⁺R⁷R⁸R⁹ (R⁷, R⁸ and R⁹ independently represent an alkyl group, respectively),
in the General Formula (Ia), R⁶ represents an alkyl group, R² and R⁶ may bind with each other to form a ring;
in the General Formula (IIa) and (IIb) shown above,
R¹¹ represents an alkyl group or an ω-aminoalkyl group,
R¹² represents a hydrogen atom or an alkyl group,
R¹³ represents an atomic group represented by a formula —(CH₂)ₘ— (m is a natural number of 10 or less),
R¹⁵ represents an atomic group represented by a formula —(CH₂)ₙ— (n is a natural number of 10 or less),
R^{N} is an atomic group represented by any one of formulae —NH₂, —NHR¹⁷, —NR¹⁷R¹⁸ and —N⁺R¹⁷R¹⁸R¹⁹ (R¹⁷, R¹⁸, R¹⁹, respectively independently represents alkyl groups),
in the General Formula (IIa), R¹⁶ represents an alkyl group, R¹² and R¹⁶ may bind with each other to form a ring.

11: The autophagy detection reagent according to claim 10, wherein the reagent comprises a fluorescent compound or a salt thereof represented by Formula 6h shown below and a fluorescent compound or a salt thereof represented by Formula 11 shown below, or
a fluorescent compound or a salt thereof represented by Formula 4b shown below and a fluorescent compound or a salt thereof represented by Formula 13 shown below. [chemical structure]
